(19) United States
(12) Patent Application Publication
(10) Pub. No.: US 2009/0099150 A1
(43) Pub. Date: Apr. 16, 2009

(54) Methotrexate Combinations For Treating Inflammatory Diseases

(76) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/814,347

(22) PCT Filed: Jan. 19, 2006
(86) PCT No.: PCT/US06/01733

§ 371 Date: Nov. 26, 2008

Related U.S. Application Data
(60) Provisional application No. 60/645,173, filed on Jan. 19, 2005.

(51) Int. Cl. A61K 31/4353; C07F 15/00

(52) U.S. Cl. 514/185; 546/10; 548/402

(57) ABSTRACT

Compounds, methods and kits for treating inflammatory diseases are disclosed. The treatment comprises administering to a patient in need thereof, methotrexate and a Reactive Oxygen Species scavenger in a pharmaceutically acceptable formulation.

### CROSS-REFERENCE TO RELATED APPLICATIONS

This application claims priority from U.S. Provisional Application Ser. No. 60/645,173 filed on Jan. 19, 2005, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

This invention relates generally to the treatment of Inflammatory diseases and, more particularly, to compositions and methods for treating inflammatory diseases with the combination of methotrexate and a Reactive Oxygen Species (ROS, including superoxide radicals and peroxynitrite) scavenger. The compositions and methods are useful in treating chronic inflammatory diseases.

2. Description of Related Art

Inflammation at the cellular level, involves a complex set of interactions among soluble factors and cells that can arise in any tissue in response to traumatic, infectious, post-ischaemic, toxic or autoimmune injury (Nathan, Nature 420:856-852, 2002). Chronic inflammation persists for a relatively long duration and can result in significant loss of function. A number of agents are believed to be useful for treating chronic inflammatory diseases. One such agent, methotrexate, has been reported to have beneficial effects in chronic inflammatory diseases such as rheumatoid arthritis (Weinblattet at, N. Engl. J. Med. 312: 818-822, 1985; Perhala et al, Comp. Ther 17:51-60, 1992; Glannini et al, N. Engl. J. Med. 326:1043-1049, 1992), psoriasis (Lebwohl, J. Am. Acad Dermatol. 45:649-661, 2001), inflammatory bowel disease (Feagan et al. Gastroenterol. Clin. N. Am. 33:407-420, 2004) and corticosteroid-dependent asthma (Mullarkey et al., N. Engl. J. Med. 318:603-607, 11988). Nevertheless, agents used for treating chronic inflammatory diseases, including methotrexate, can produce unwanted side effects (Weinblaft, N. Engl. J. Med. 332:330-331, 1995).

Recently, a superoxide dismutase mimetic, M40403, was shown to be effective in an animal models of inflammation (Salvemini et al., Science 286:304-306, 1999) and, more specifically, in an animal model of rheumatoid arthritis: (Salvemini et al., Arthritis & Reumatism 44:2909-2921, 2001). Nevertheless, the comparative efficacy of this agent by itself or in combination with other anti-inflammatory agents such as methotrexate has neither been reported nor suggested.

### BRIEF SUMMARY OF THE INVENTION

Accordingly, it is an object of the invention to overcome these and other problems associated with the related art. These and other objects, features and technical advantages are achieved by providing synergistic methotrexate and ROS scavenger combinations for the enhanced treatment of inflammatory diseases or disorders.

This invention provides a method for treating an inflammatory disease, the method comprising administering to a patient in need thereof, methotrexate and a ROS scavenger in a pharmaceutically acceptable formulation. In one alternative, the ROS scavenger is a superoxide dismutase mimetic. In accordance with one aspect of the invention, the superoxide dismutase mimetic is represented by the formula:

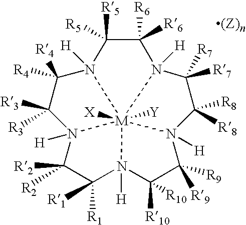

wherein

(i) R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀ are independently:

(i^{a}) hydrogen; or

(i^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(i^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(ii) optionally, one or more of R₁ or R′₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ or R′₁₀, together with the carbon atoms to which they are attached independently form a substituted or unsubstituted and saturated, partially saturated, or unsaturated cycle or heterocycle having 3 to 20 carbon atoms; and

(iii) optionally, one or more of R₁ and R′₁, R₂ and R′₂, R₃ and R′₃, R₄ and R′₄, R₅ and R′₅, R₆ and R′₆, R₇ and R′₇, R₈ and R′₈, R₉ and R′₉, and R₁₀ and R′₁₀, together with the carbon atom to which they are attached independently form a substituted or unsubstituted and saturated, partially saturated, or unsaturated cycle or heterocycle having 3 to 20 carbon atoms; and

(iv) optionally, one or more of R₁₀ or R′₁₀ and R₁ or R′₁, R₂ or R′₂ and R₃ or R′₃, R⁴ or R′₄, and R₅ or R′₅, R₆ or R′₆ and R₇ or R′₇, or R₈ or R′₈ and R⁹ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(v) optionally, one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, together with a different one of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vi) combinations of any of (i) through (v) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀; and

n is an integer from 0 to 3.

Preferably, M is selected from the group consisting of Mn, Fe, Ni, Cu and V. In an alternative, the superoxide dismutase mimetic is represented by the formula:

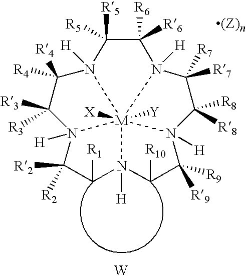

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) one or more of R₁, R₂, R′₂, R₃, R′₃, R₄, R′₄, R₅, R′₅, R₆, R′₆, R₇, R′₇, R₈, R′₈, R₉, R′₉, and R₁₁ are independently:

(ii^{a}) hydrogen; or

(ii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(ii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(iii) optionally, one or more of R₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ together with the carbon atoms to which they are attached independently form a substituted or unsubstituted and saturated, partially saturated, or unsaturated cycle or heterocycle having 3 to 20 carbon atoms, and

(iv) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted and saturated, partially saturated, or unsaturated cycle or heterocycle having 3 to 20 carbon atoms; and

(v) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vi) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, together with a different one of R₁, R₂, R′₂, R₃, R′₃, R₄, R′₄, R₅, R′₅, R₆, R′₆, R₇, R′₇, R₈, R′₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vii) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, may be bound to an atom of heterocycle W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) combinations of any of (i) through (vii) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic-acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlonte, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R₂, R′₂, R₃, R′₃, R₄, R′₄, R₅, R′₅, R₆, R′₆, R₇, R′₇, R₈, R′₈, R₉, R′₉, and R₁₀; and

n is an integer from 0 to 3.

Preferably, M is selected from the group consisting of Mn, Fe, Ni, Cu and V, and W is a substituted or unsubstituted pyridino moiety.

In yet another alternative, the superoxide dismutase mimetic is represented by the formula:

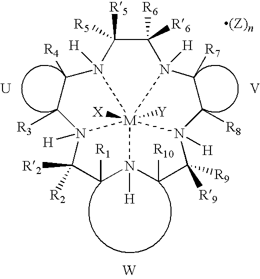

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) two sets of two adjacent carbon atoms of the macrocycle independently form substituted or unsubstituted, saturated, partially saturated or unsaturated, cycles or heterocycles U and V having 3 to 20 carbon atoms; and

(iii) R₁, R₂, R′₂, R₃, R₄, R₅, R′₅, R₆, R′₆, R₇, R₈, R′₉, and R₁₀ are independently:

(iii^{a}) hydrogen; or

(iii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(iii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(iv) optionally, one or more of R₁ and R₂ or R′₂, R₅ or R′₅ and R₆ or R′₆, R₉ or R′₉ and R₁₀ together with the carbon atoms to which they are attached independently form a substituted or unsubstituted and saturated, partially saturated, or unsaturated cycle or heterocycle having 3 to 20 carbon atoms; and

(v) optionally, one or more of R₂ and R′₂, R₅ and R′₅, R₆ and R′₆, and R₉ and R₁₀, together with the carbon atom to which they are attached independently form a substituted or unsubstituted and saturated, partially saturated, or unsaturated cycle or heterocycle having 3 to 20 carbon atoms; and

(vi) optionally, one or more of R₂ or R′₂ and R₃, R₄ and R₅ or R′₅, R₆ or R′₆ and R₇, or R₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vii) optionally, one or more of R₁, R₂, R′₂, R₃, R₄, R₅; R′₅, R₆, R′₆, R₇, R₈, R₉, R′₉, and R₁₀, together with a different one of R₁, R₂, R′₂, R₃, R₄, R₅, R′₅, R₆, R′₆, R₇, R₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) optionally, one or more of R₁, R₂, R′₂, R₃, R₄, R₅, R′₅, R₆, R′₆, R₇, R₈, R₉, R′₉, and R₁₀, may be individually bound to an atom of heterocycles U, V and W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(ix) combinations of any of (i) through (viii) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfinic acid, alkyl sulfinic acid, aryl sulfinic-acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic, acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R₂, R′₂, R₃, R₄, R₅, R′₅, R₆, R′₆, R₇, R₈, R₉, R′₉, and R₁₀; and

n is an integer from 0 to 3.

Preferably, M is selected from the group consisting of Mn, Fe, Ni, Cu and V. In accordance with a further aspect of the invention, U and V are saturated cycloalkyl heterocycles having 3 to 20 carbon atoms preferably saturated cycloalkyl heterocycles having 4 to 10 carbon atoms, and still more preferably U and V are trans-cyclohexanyl fused rings. In yet another aspect of the present invention, W is a substituted or unsubstituted pyridino moiety, more preferably, U and V are trans-cyclohexanyl fused rings and W is a substituted pyridino moiety. Preferably, the superoxide dismutase mimetic is. represented by the formula:

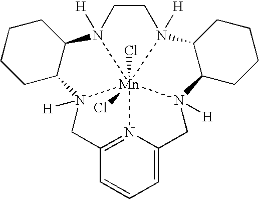

In yet another aspect of the present invention, the ROS scavenger is a peroxynitrite scavenger. Preferably, the peroxynitrite scavenger is represented by a formula selected from the group of formulas consisting of:

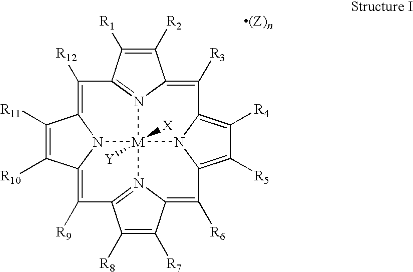

wherein R₃, R₆, R₉ and R₁₂ are independently selected from the group consisting of H, alkyl, alkenyl, CH₂, COOH, phenyl, pyridyl, and N-alkylpyridyl, such that phenyl, pyridyl and N-alkylpyridyl are:

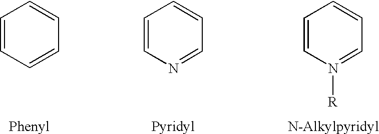

which are attached at a carbon atom; and

wherein Phenyl is optionally substituted by a substituent selected from the group consisting of a halogen, alkyl, aryl, benzyl, COOH, CONH₂, SO₃H, NO₂, NH₂, N(R)³⁺ and NHCOR′, wherein R is selected from the group consisting of hydrogen, alkyl, aryl and alkaryl, and R′ is alkyl;

wherein Pyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and

wherein N-Alkylpyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and

wherein R₁, R₂, R₄, R₅, R₇, R₈, R₁₀, or R₁₁ are independently selected from the group consisting of H, alkyl, alkenyl, carboxyalkyl, Cl, Br, F, NO₂, hydroxyalkyl, and SO₃H; and further wherein R₁ and R₂ optionally form a heterocycle having 5 to 8 carbon atoms and form a ring with the carbon atoms of the macrocycle to which they are attached;

X and Y are ligands or charge-neutralizing anions which are derived from any monodentate or polydentate coordinating ligand or ligand system or the corresponding anion thereof and are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl, amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile; aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroanitronate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins; with the proviso that when the X and Y containing complex has a net positive charge then Z is a counter ion which is independently selected from the group consisting of X and Y, or when the X and Y containing complex has net negative charge then Z is a counter ion selected from a group consisting of alkaline and alkaline earth cations, organic cations such as alkyl or alkylaryl ammonium cations; and

M is selected from the group consisting of Mn, Fe, Ni and V; and

n is an integer from 0 to 4.

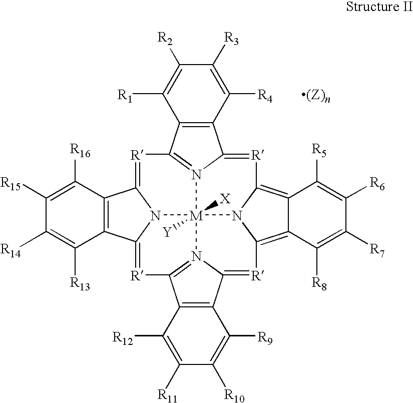

wherein R′ is CH or N;

R₁, R₂, R₃, R₄, R₅, R₆, R₇, R₈, R₉, R₁₀, R₁₁, R₁₂, R₁₃, R₁₄, R₁₅, and R₁₆, are independently selected from the group consisting of H, SO₃H, COOH, NO₂, NH₂, and N-alkylamino; and

X, Y, Z, M and n are as defined above;

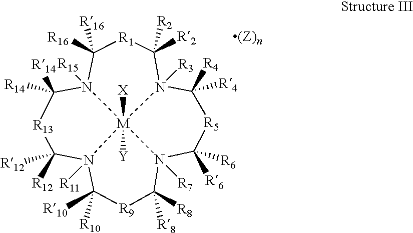

A

wherein R₁, R₅, R₉, and R₁₃ are independently selected from the group consisting of a direct bond and CH₂;

R₂, R′₂, R₄, R′₄, R₆, R′₆, R₈, R′₈, R₁₀, R′₁₀, R₁₂, R′₁₂, R₁₄, R′₁₄, R₁₆, and R′₁₆ are independently selected from the group consisting of H and alkyl;

R₃, R₇, R₁₁, and R₁₅ are independently selected from the group consisting of H and alkyl; and

X, Y, Z, M and n are as defined above;

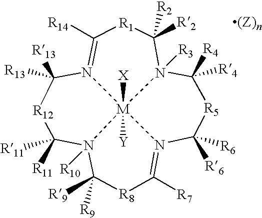

B

wherein R₁, R₅, R₈, and R₁₂ are independently selected from the group consisting of a direct bond and CH₂;

R₂, R′₂, R₄, R′₄, R₆, R′₆, R₇, R₉, R′₉, R₁₁, R′₁₁, R₁₃, R′₁₃, and R₁₄ are independently selected from the group consisting of H and alkyl;

R₃ and R₁₀ are independently selected from the group consisting of H and alkyl; and

X, Y, Z, M and n are as defined above;

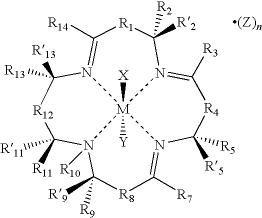

C

wherein R₁, R₄, R₈, and R₁₂ are independently selected from the group consisting of a direct bond and CH₂;

R₂, R′₂, R₃, R₅, R′₅, R₇, R₉, R′₉, R₁₁, R′₁₁, R₁₃, R′₁₃ and R₁₄ are independently selected from the group consisting of H and alkyl;

R₁₀ is H or alkyl; and

X, Y, Z, M and n are as defined above;

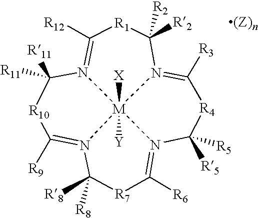

D

wherein R₁, R₄, R₇ and R₁₀ are independently selected from the group consisting of a direct bond and CH₂;

R₂, R′₂, R₃, R₅, R′₅, R₆, R₈, R′₉, R₁₁, R′₁₁ and R₁₂ are independently selected from the group consisting of H and alkyl; and

X, Y, Z, M and n are as defined above;

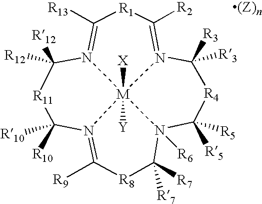

E

wherein R₁, R₄, R₈ and R₁₁ are independently selected from the group consisting of a direct bond and CH₂;

R₂, R₃, R′₃, R₅, R′₅, R₇, R′₇, R₉, R₁₀, R′₁₀, R₁₂, R′₁₂, and R₁₃ are independently selected from the group consisting of H and alkyl;

R₆ is hydrogen or alkyl; and

X, Y, Z, M and n are as defined above;

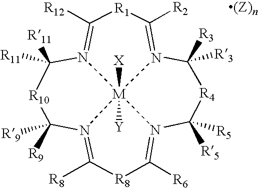

F

wherein R₁, R₄, R₇ and R₁₀ are independently selected from the group consisting of H and alkyl;

R₂, R₃, R′₃, R₅, R′₅, R₆, R₈, R₉, R′₉, R₁₁, R′₁₁ and R₁₂ are independently selected from the group consisting of H and alkyl; and

X, Y, Z, M and n are as defined above;

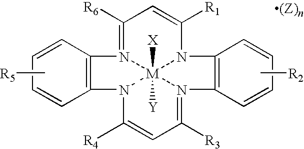

G

wherein R₁, R₃, R₄, and R₆ are independently selected from the group consisting of H and alkyl;

R₂ and R₅ are independently selected from the group consisting of H, alkyl, SO₃H, NO₂, NH₂, halogen, COOH and N(R)³⁺ wherein R is as defined above; and

X, Y, Z, M and n are as defined above;

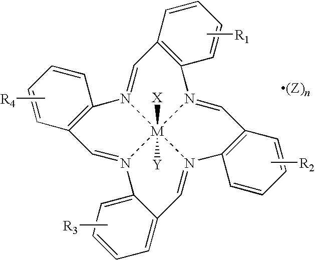

H

wherein R₁, R₂, R₃, and R₄ are independently selected from the group consisting of H, alkyl, SO₃H, NO₂, NH₂, halogen, COOH and N(R)³⁺ wherein R is as defined above; and

X, Y, Z, M and n are as defined above; and

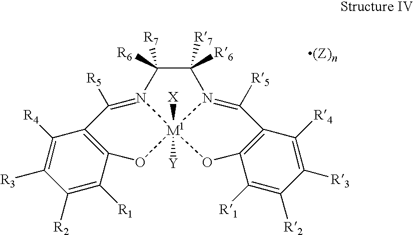

wherein R₁, R′₁, R₂, R′₂, R₃, R′₃, R₄, R′₄, R₅, R′₅, R₆, R′₆, R₇ and R′₇ are independently selected from the group consisting of H, alkyl, alkoxy, NO₂, aryl, halogen, NH₂ and SO₃H, further wherein R₆, R′₆, R₇ and R₁₇ together with one other of R₆, R′₆, R₇ and R′₇ optionally form a heterocycle having 5 to 8 carbon atoms and form a ring with the carbon atoms of the macrocycle to which they are attached;

M¹ is selected from the group consisting of Fe, Ni or V; and

X, Y, Z and n are as defined above.

In a further aspect of the present invention, the methotrexate and the ROS scavenger are administered in amounts that act synergistically in treating the inflammatory disease. The methotrexate can be administered in an amount of at most 0.015 mg/kg, or preferably at most 2 mg/kg. In yet another aspect, the pharmaceutically acceptable formulation is a pharmaceutically acceptable oral formulation and administering comprises administering orally. Preferably, the patient is a human patient and the inflammatory disease is rheumatoid arthritis, psoriasis, inflammatory bowel disease or orticosteroid-dependent asthma. In one alternative, the methotrexate and the ROS scavenger are administered in one compound. In another alternative, the methotrexate and the ROS scavenger are administered as separate compositions.

This invention provides a pharmaceutical compound for treating an inflammatory disease, the compound comprising methotrexate and a ROS scavenger in a pharmaceutically acceptable formulation containing at least one unit dose suitable for administration to a patient in need thereof. According to one aspect of the present invention, the ROS scavenger is a superoxide dismutase mimetic. In one alternative, the superoxide dismutase mimetic is represented by the formula:

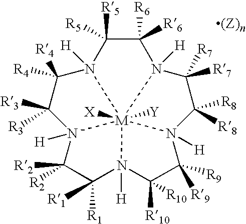

wherein

(i) R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀ are independently:

(i^{a}) hydrogen; or

(i^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(i^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(ii) optionally, one or more of R₁ or R′₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ or R′₁₀ together with the carbon atoms to which they are attached independently form a substituted or unsubstituted and saturated, partially saturated, or unsaturated cycle or heterocycle having 3 to 20 carbon atoms; and

(iii) optionally, one or more of R₁ and R′₁, R₂ and R′₂, R₃ and R′₃, R₄ and R′₄, R₅ and R′₅, R₆ and R′₆, R₇ and R′₇, R₈ and R′₈, R₉ and R′₉, and R₁₀ and R′₁₀, together with the carbon atom to which they are attached independently form a substituted or unsubstituted and saturated, partially saturated, or unsaturated cycle or heterocycle having 3 to 20 carbon atoms; and

(iv) optionally, one or more of R₁₀ or R′₁₀ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(v) optionally, one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, together with a different one of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vi) combinations of any of (i) through (v) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀; and

n is an integer from 0 to 3.

Preferably, M is selected from the group consisting of Mn, Fe, Ni, Cu and V.

In another alternative, the superoxide dismutase mimetic is represented by the formula:

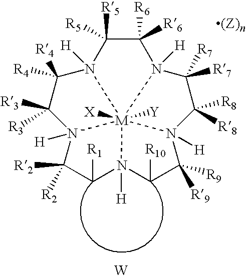

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20-carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) one or more of R₁, R₂, R′₂, R₃, R′₃, R₄, R′₄, R₅, R′₅, R₆; R′₆, R₇, R′₇, R₈, R′₈, R₉, R′₉, and R₁₀ are independently:

(ii^{a}) hydrogen; or

(ii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(ii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(iii) optionally, one or more of R₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ together with the carbon atoms to which they are attached independently form a substituted or unsubstituted and saturated, partially saturated, or unsaturated cycle or heterocycle having 3 to 20 carbon atoms; and

(iv) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted and saturated, partially saturated, or unsaturated cycle or heterocycle having 3 to 20 carbon atoms; and

(v) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vi) optionally, one or more of R₁, R₂, R′₂, R₃, R′₃, R₄, R′₄, R₅, R′₅, R₆, R′₆, R₇, R′₇, R₆, R′₈, R₉, R′₉, and R₁₀, together with a different one of R₁, R₂, R′₂, R₃, R′₃, R₄, R′₄, R₅, R′₅, R₆, R′₆, R₇, R′₇, R₈, R′₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vii) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, may be bound to an atom of heterocycle W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) combinations of any of (i) through (vii) above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R₂, R′₂, R₃, R′₃, R₄, R′₄, R₅, R′₅, R₆, R′₆, R₇, R′₇, R₈, R′₈, R₉, R′₉, and R₁₀; and

n is an integer from 0 to 3.

Preferably, M is selected from the group consisting of Mn, Fe, Ni, Cu and V and W is a substituted or unsubstituted pyridino moiety.

In yet another alternative, the superoxide dismutase mimetic is represented by the formula:

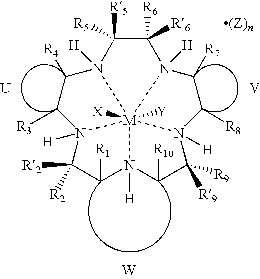

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) two sets of two adjacent carbon atoms of the macrocycle independently form substituted or unsubstituted; saturated, partially saturated or unsaturated, cycles or heterocycles U and V having 3 to 20 carbon atoms; and

(iii) R₁, R₂, R′₂, R₃, R₄, R₅, R′₅, R₆, R′₆, R₇, R₈, R₉, R′₉, and R₁₀ are independently:

(iii^{a}) hydrogen; or

(iii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(iii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), arid substituents attached to the α-carbon of α-amino acids, wherein R₁₁ and R′₂ are independently hydrogen or alkyl; and

(iv) optionally, one or more of R₁ and R₂ or R′₂, R₅ or R′₅ and R₆ or R′₆, R₉ or R′₉, and R₁₀ together with the carbon atoms to which they are attached independently form a substituted or unsubstituted and saturated, partially saturated, or unsaturated cycle or heterocycle having 3 to 20 carbon atoms; and

(v) optionally, one or more of R₂ and R′₂, R₅ and R′₅, R₆ and R′₆, and R₉ and R′₉, together with the carbon atom to which they are attached-independently form a substituted or unsubstituted and saturated, partially saturated, or unsaturated cycle or heterocycle having 3 to 20 carbon atoms; and

(vi) optionally, one or more of R₂ or R′₂, and R₃, R₄ and R₅ or R′₅, R₆ or R′₆ and R₇, or R₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vii) optionally, one or more of R₁, R₂, R′₂, R₃, R₄, R₅, R′₅, R₆, R′₆, R₇, R₈, R₉, R′₉, and R₁₀, together with a different one of R₁, R₂, R′₂, R₃, R₄, R₅, R′₅, R₆, R′₆, R₇, R₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) optionally, one or more of R₁, R₂, R′₂, R₃, R₄, R₅, R′₅, R₆, R′₆, R₇, R₈, R₉, R′₉, and R₁₀, may be individually bound to an atom of heterocycles, U, V and W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(ix) combinations of any of (i) through (viii), above;

wherein

M is a transition metal;

X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic-acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine-sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R₂, R′₂, R₃, R₄, R₅, R′₅, R₆, R′₆, R₇, R₈, R₉, R′₉, and R₁₀; and

n is an integer from 0 to 3.

Preferably, M is selected from the group consisting of Mn, Fe, Ni, Cu and V and U and V are saturated cycloalkyl heterocycles having 3 to 20 carbon atoms, more preferably saturated cycloalkyl heterocycles having 4 to 10 carbon atoms, and still more preferably U and V are trans-cyclohexanyl fused rings. In accordance with a further aspect of the present invention, W is a substituted or unsubstituted pyridino moiety, more preferably U and V are trans-cyclohexanyl fused rings and W is a substituted pyridino moiety. Preferably, the superoxide dismutase mimetic is represented by the formula:

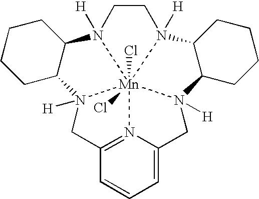

In yet another aspect of the present invention, the ROS scavenger is a peroxynitrite scavenger. Preferably, the peroxynitrite scavenger is represented by a formula selected from the group of formulas consisting of:

Structure I

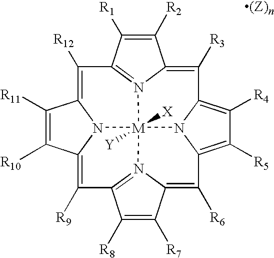

wherein R₃, R₆, R₉ and R₁₂ are independently selected from the group consisting of H, alkyl, alkenyl, CH₂, COOH, phenyl, pyridyl, and N-alkylpyridyl, such that phenyl, pyridyl and N-alkylpyridyl are:

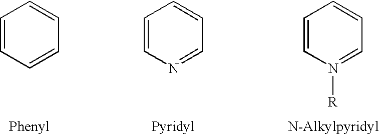

which are attached at a carbon atom; and

wherein Phenyl is optionally substituted by a substituent selected from the group consisting of a halogen, alkyl, aryl, benzyl, COOH, CONH₂, SO₃H, NO₂, NH₂, N(R)³⁺ and NHCOR′, wherein R is selected from the group consisting of hydrogen, alkyl, aryl and alkaryl, and R′ is alkyl:

wherein Pyridyl is optionally substituted by a substituent selected from the group consisting of a halogen, alkyl aryl, benzyl, COOH, CONH₂, SO₃H, NO₂, NH₂, N(R)³⁺ and NHCOR′, wherein R and R′ are as defined above; and

wherein N-Alkylpyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and

wherein R₁, R₂, R₄, R₅, R₇, R₈, R₁₀, or R₁₁ are independently selected from the group consisting of H, alkyl, alkenyl, carboxyalkyl, Cl, Br, F, NO₂, hydroxyalkyl, and SO₃H; and further wherein R₁ and R₂ optionally form a heterocycle having 5 to 8 carbon atoms and form a ring with the carbon atoms of the macrocycle to which they are attached;

X and Y are ligands or charge-neutralizing anions which are derived from any monodentate or polydentate coordinating ligand or ligand system or the corresponding anion thereof and are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl, amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroanitmonate, hypophosphite, iodate, periodate, metaborate, tetraaryl borate; tetra alkyl borate, tartrate, salicylate, succinate, citrate, ascorbate, saccharinate, amino acid, hydroxamic acid, thiotosylate, and anions of ion exchange resins; with the proviso that when the X and Y containing complex has a net positive charge then Z is a counter ion which is independently selected from the group consisting of X and Y, or when the X and Y containing complex has net negative charge then Z is a counter ion selected from a group consisting of alkaline and alkaline earth cations, organic cations such as alkyl or alkylaryl ammonium cations; and

M is selected from the group consisting of Mn, Fe, Ni and V; and

n is an integer from 0 to 4.

Structure II

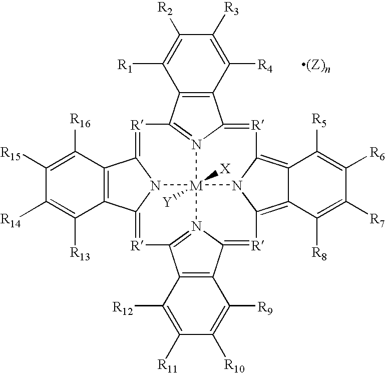

wherein R′ is CH or N;

R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of H, SO₃H, COOH, NO₂, NH₂, and N-alkylamino; and

X, Y, Z, M and n are as defined above;

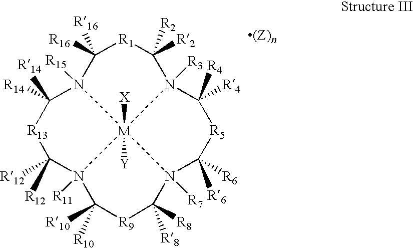

A

wherein R₁, R₅, R₉, and R₁₃ are independently selected from the group consisting of a direct bond and CH₂;

R₂, R′₂, R₄, R′₄, R₆, R′₆, R₈, R′₈, R₁₀, R′₁₀, R₁₂, R′₁₂, R₁₄, R′₁₄, R₁₆, and R′₁₆ are independently selected from the group consisting of H and alkyl;

R₃, R₇, R₁₁, and R₁₅ are independently selected from the group consisting of H and alkyl; and

X, Y, Z, M and n are as defined above;

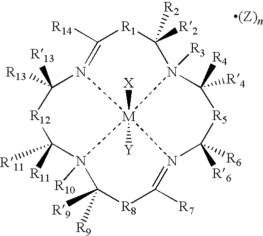

B

wherein R₁, R₅, R₈, and R₁₂ are independently selected from the group consisting of a direct bond and CH₂;

R₂, R′₂, R₄, R′₄, R₆, R′₆, R₇, R₉, R′₉, R₁₁, R′₁₁, R₁₃, R′₁₃, and R₁₄ are independently selected from the group consisting of H and alkyl;

R₃ and R₁₀ are independently selected from the group consisting of H and alkyl; and

X, Y, Z, M and n are as defined above;

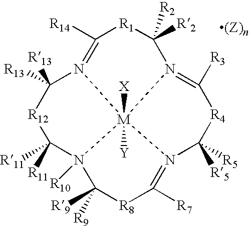

C

wherein R₁, R₄, R₈, and R₁₂ are independently selected from the group consisting of a direct bond and CH₂;

R₂, R′₂, R₃, R₅, R′₅, R₇, R₉, R′₉, R₁₁, R′₁₁, R₁₃, R′₁₃ and R₁₄ are independently selected from the group consisting of H and alkyl;

R₁₀ is H or alkyl; and

X, Y, Z, M and n are as defined above;

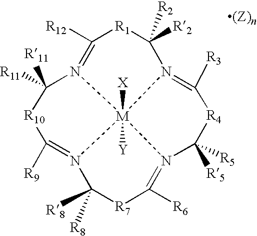

D

wherein R₁, R₄, R₇ and R₁₀ are independently selected from the group consisting of a direct bond and CH₂;

R₂, R′₂, R₃, R₅, R′₅, R₆, R₈, R′₈, R₉, R₁₁, R′₁₁ and R₁₂ are independently selected from the group consisting of H and alkyl; and

X, Y, Z, M and n are as defined above;

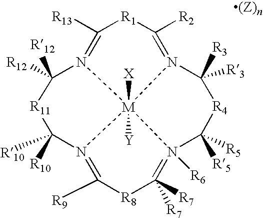

E

wherein R₁, R₄, R₈ and R₁₁ are independently selected from the group consisting of a direct bond and CH₂;

R₂, R₃, R′₃, R₅, R′₅, R₇, R′₇, R₉, R₁₀, R′₁₀, R₁₂, R′₁₂ and R₁₃ are independently selected from the group consisting of H and alkyl;

R₆ is hydrogen or alkyl; and

X, Y, Z, M and n are as defined above;

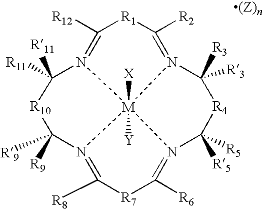

F

wherein R₁, R₄, R₇ and R₁₀ are independently selected from the group consisting of H and alkyl;

R₂, R₃, R′₃, R₅, R′₅, R₆, R₈, R₉, R′₉, R₁₁, R′₁₁ and R₁₂ are independently selected from the group consisting of H and alkyl; and

X, Y, Z, M and n are as defined above;

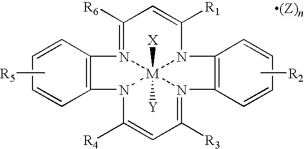

G

wherein R₁, R₃, R₄, and R₆ are independently selected from the group consisting of H and alkyl;

R₂ and R₅ are independently selected from the group consisting of H, alkyl, SO₃H, NO₂, NH₂, halogen, COOH and N(R)³⁺ wherein R is as defined above; and

X, Y, Z, M and n are as defined above;

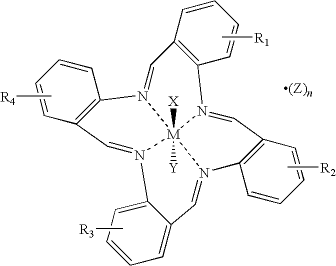

H

wherein R₁, R₂, R₃, and R₄ are independently selected from the group consisting of H, alkyl, SO₃H, NO₂, NH₂, halogen, COOH and N(R)³⁺ wherein R is as defined above; and

X, Y, Z, M and n are as defined above; and

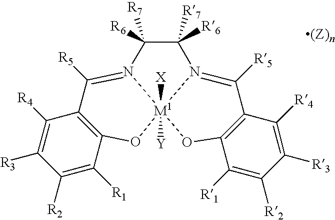

wherein R₁, R′₁, R₂, R′₂, R₃, R′₃, R₄, R′₄, R₅, R′₅, R₆, R′₆, R₇ and R′₇ are independently selected from the group consisting of H, alkyl, alkoxy, NO₂, aryl, halogen, NH₂ and SO₃H, further wherein R₆, R′₆, R₇ and R′₇ together with one other of R₆, R′₆, R₇ and R′₇ optionally form a heterocycle having 5 to 8 carbon atoms and form a ring with the carbon atoms of the macrocycle to which they are attached;

M¹ is selected from the group consisting of Fe, Ni or V; and

X, Y, Z and n are as defined above.

In a further aspect of the present invention, the methotrexate and the ROS scavenger are administered in amounts that act-synergistically in treating the inflammatory disease. The methotrexate can be administered in an amount of at most 0.015 mg/kg, or preferably at most 2 mg/kg. In yet another aspect, the pharmaceutically acceptable formulation is a pharmaceutically acceptable oral formulation and administering comprises administering orally. Preferably, the patient is a human patient and the inflammatory disease is rheumatoid arthritis, psoriasis, inflammatory bowel disease or corticosteroid-dependent asthma.

In yet another aspect of the present invention, a kit is provided for treating an inflammatory disease, the kit comprising methotrexate and a ROS scavenger, wherein each of the methotrexate and the ROS scavenger are present in a pharmaceutically acceptable formulation containing at least one unit dose suitable for administration to a patient in need thereof.

These and other features, aspects and advantages of the present invention will become better understood with reference to the following description, examples and appended claims.

### BRIEF DESCRIPTION OF THE SEVERAL VIEWS OF THE DRAWINGS

FIG. 1 illustrates the effects of no treatment (CIA), treatment with M40403 alone at 2 mg/kg, treatment with methotrexate (MET) alone at 0.015 mg/kg and 0.15 mg/kg and treatment with the methotrexate (0.015 mg/kg) and M40403 (2 mg/kg) in combination, on the onset of collagen-induced arthritis (CIA) in rats showing (A) the percentage of arthritic rats showing clinical scores of arthritis under the various treatment regimes and (B) the median arthritic score effect for the various treatment regimes with values significantly different from that in animals receiving no treatment indicated with asterisks.

FIG. 2 illustrates the effect of no treatment control (CIA), treatment with M40403 alone at 2 mg/kg, treatment with methotrexate (MET) alone at 0.015 and 0.15 mg/kg, and treatment with methotrexate (0.015 mg/kg) and M40403 (2 mg/kg) in combination, on secondary lesion in animals receiving type II collagen to produce collagen-induced arthritis with values significantly different from that in animals receiving no treatment indicated with asterisks.

FIG. 3 illustrates the representative histology of the paw of (A) a control animal, (B) an animal receiving with type II collagen to produce collagen-induced arthritis (CIA) and no treatment, (C) an animal receiving type II collagen and treatment with methotrexate (0.15 mg/kg), and (D) an animal receiving type II collagen and treatment with methotrexate (0.015 mg/kg) and M40403 (2 mg/kg) in combination.

FIG. 4 illustrates the mean (±s.e., n=10) effects of vehicle, treatment with M40403 alone at 2 mg/kg, treatment with methotrexate (MET) alone at 0.015 mg/kg. and 0.15 mg/kg, and treatment with methotrexate (0.015 mg/kg) and M40403 (2 mg/kg) in combination on (A) histological damage score and (B) radiograph score in animals immunized with type II collagen to produce collagen-induced arthritis with values significantly different (p<0.01) from that of sham-treated animal indicated by asterisks and values significantly different (p<0.01) from that in animals receiving vehicle indicated with degree sign.

FIG. 5 illustrates the radiographic progression of collagen-induced arthritis (CIA) showing (A) no evidence of pathology in the Uibiotarsal joints of normal rats, (B) bone resorption (arrow) in the hind paws of collagen-type II immunized rats at 35 days, and suppression of joint pathology in (C) collagen-type II immunized rats receiving treatment with methotrexate (MET) at 0.15 mg/kg and (D) collagen-type II immunized rats receiving treatment with methotrexate (0.015 mg/kg) and M40403 (2 mg/kg) in combination.

FIG. 6 illustrates the mean (±s.e., n=10) plasma levels of TNF-α and IL-1β in animals receiving vehicle and in animals immunized with type II collagen and receiving no treatment, treatment with M40403 (2 mg/kg) alone, treatment with methotrexate alone (0.015 mg/kg and 0.15 mg/kg) and treatment with methotrexate (0.015 mg/kg) and M40403 (2 mg/kg) in combination with significant values (P<0.01) compared to that in vehicle-treated group indicated by asterisks and significant values (P>0.01) compared to that in animals immunized with type II collagen and receiving no treatment indicated by degree sign.

FIG. 7 illustrates the mean (±s.e., n=10) effect on body weight gain in sham treated animals and in animals immunized with type II collagen and receiving no treatment, treatment with M40403 (2 mg/kg) alone, treatment with methotrexate alone (0.015 mg/kg and 0.15 mg/kg) and treatment with methotrexate (0.015 mg/kg) and M40403 (2 mg/kg) in combination with significant values (P<0.01) compared to that in vehicle-treated group indicated by asterisks and significant values (P>0.01) compared to that in animals immunized with type II collagen and receiving no treatment indicated by degree sign.

### DETAILED DESCRIPTION OF THE INVENTION

### Abbreviations and Definitions

To facilitate understanding of the invention, a number of terms and abbreviations as used herein are defined below as follows:

The term “alkenyl”, alone or in combination, means an alkyl-substituent having one or more double bonds. Examples of such alkenyl substituents include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkyl”, alone or in combination, means a straight-chain or branched-chain alkyl substituent containing from 1 to about 22 carbon atoms, preferably from about 1 to about 18 carbon atoms, and most preferably from about 1 to about 12 carbon atoms. Examples of such substituents include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The terms “alkylcycloalkyl” and “alkenylcycloalkyl” mean a cycloalkyl substituent as defined above which is substituted by an alkyl or alkenyl substituent as defined above. Examples of alkylcycloalkyl and alkenylcycloalkyl substituents include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl substituent as defined above which is substituted by an alkyl or alkenyl substituent as defined above. Examples of alkylcycloalkenyl and alkenylcycloalkenyl substituents include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “alkynyl”, alone or in combination, means an alkyl substituent having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “aralkyl”, alone or in combination, means an alkyl or cycloalkyl substituent as defined above in which one hydrogen atom is replaced by an aryl substituent as defined above, such as benzyl, 2-phenylethyl, and the like.

The term “aryl”, alone or in combination, means a phenyl or naphthyl substituent which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “cycloalkenyl”, alone or in combination, means a cycloalkyl substituent having one or more double bonds. Examples of cycloalkenyl substituents include, but are not limited to, cyclopentenyl, cyclohexenyl, cyclooctenyl, cyclopentadienyl, cyclohexadienyl and cyclooctadienyl.

The terms “cyclic”, “cycle” or “cycylyl” means a ring structure containing 3 to 20 carbon atoms, preferably 5 to 10 carbon atoms, which may be heterocyclic. The cyclic, cycle or cycylyl can also contain more than one ring.

The term “cycloalkenylalkyl” means an alkyl substituent as defined above which is substituted by a cycloalkenyl substituent as defined above. Examples of cycloalkenylalkyl substituents include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-1-yl)hexyl, 1-(1-cyclopenten-1-yl)nonyl and 1-(1-cyclohexen-1-yl)nonyl.

The term “cycloalkyl”, alone or in combination means a cycloalkyl radical containing from 3 to about 10, preferably from 3 to about 8, and most preferably from 3 to about 6, carbon atoms. Examples of such cycloalkyl substituents include, but are not limited to, cyclopropyl, cyclobutyl, cyclopentyl, cyclohexyl, cycloheptyl, cyclooctyl, and perhydronaphthyl.

The term “cycloalkylalkyl” means an alkyl substituent as defined above which is substituted by a cycloalkyl substituent as defined above. Examples of cycloalkylalkyl substituents include, but are not limited to, cyclohexylmethyl, cyclopentylmethyl, (4-isopropylcyclohexyl)methyl, (4-t-butyl-cyclohexyl)methyl, 3-cyclohexylpropyl, 2-cyclohexylmethylpentyl, 3-cyclopentylmethylhexyl, 1-(4-neopentylcyclohexyl)methylhexyl, and 1-(4-isopropylcyclohexyl)methylheptyl.

The term “cycloalkylcycloalkyl” means a cycloalkyl substituent as defined above which is substituted by another cycloalkyl substituent as defined above. Examples of cycloalkylcycloalkyl substituents include, but are not limited to, cyclohexylcyclopentyl and cyclohexylcyclohexyl.

The term “halide” means chloride, fluoride, iodide, or bromide.

The term “heterocyclic”, “heterocycle” or “heterocycylyl” means a cyclic, cycle or cycylyl containing at least one other kind of atom, in addition to carbon, in the ring. Such atoms include, but are not limited to, nitrogen, oxygen and sulfur. The heterocyclic can also contain more than one ring. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “methotrexate” as used herein, is intended to refer to the compound, (N-[4-[[(2,4-Diamino-6-pteridinyl)methyl]methylamino]benzoyl]-L-glutamic acid) including anhydrous and hydrous forms, in particular, methotrexate monohydrate and acid and salt forms such as alkali metal or alkaline earth metal salts, in particular, the disodium salt. Derivatives of methotrexate are also known in the art such as those described in U.S. Pat. Nos. 5,382,582, 5,698,556, 5,728,692 and 6,559,149, incorporated herein by reference in their entirety. Such derivatives can also be used in combination with a nonpeptidal mimic of superoxide dismutase in the treatment of an inflammatory disease.

The term “nitrogen containing heterocycle” means a ring structure in which 2 carbons and a nitrogen of the ring are shared with the fifteen-membered macrocyclic ligand. The nitrogen containing heterocycle can contain 2 to 20, preferably 4 to 10, carbon atoms, can be substituted or unsubstituted, saturated, partially saturated or unsaturated, and can also contain nitrogen, oxygen and/or sulfur atoms in the portion of the ring which is not also part of the fifteen-membered macrocyclic ligand.

The term “R groups” means the group of variable substituents designated as “R” attached to the carbon atoms of the macrocycle, i.e., R₁, R′₁, R₂, R′₂, R₃, R′₃, R₄, R′₄, R₅, R′₆, R₆, R′₆, R₇, R′₇, R₈, R′₈, R₉, R′₉, R₁₀, and R′₁₀.

The term “saturated, partially saturated or unsaturated cycle or heterocycle” means a fused ring structure in which 2 carbons of the ring are also part of the fifteen-membered macrocyclic ligand in which the ring can contain no double bonds, (in the case of a saturated ring structure) or at least one double bond, which may be conjugated or unconjugated with another double bond. The ring structure can contain 3 to 20 carbon atoms, preferably 5 to 10 carbon atoms, which may be heterocyclic. The cyclic can also contain more than one ring.

Methotrexate Combinations for Treating Inflammatory Diseases

The present invention involves the administration of methotrexate and a ROS. scavenger in the treatment of an inflammatory disease to produce an additive or synergistic effect in which either or both agents can be administered to a patient at a lower dose to achieve the same level of efficacy.

The ROS scavengers of the present invention include superoxide dismutase mimetics that act like superoxide dismutase to catalyze the conversion of superoxide radical, O₂⁻, to molecular oxygen and hydrogen peroxide. Such compositions act like native superoxide dismutase enzymes to reduce cell injury produced by enhanced production of superoxide radical in diseases involving oxidative stress such as inflammation (Salvemini et al, Arthritis & Rheumatism 44:2909-2921, 2001). In addition, the ROS scavengers of the present invention include peroxynitrite scavengers. Such compositions catalyze the decomposition of peroxynitrite, produced by the reaction of superoxide and NO, which has been shown to play a role in cytotoxicity. The ROS scavengers of the present invention can be pentaaza-macrocyclic complexes, porphyrin complexes, salen complexes, and more specifically, those compositions as disclosed in U.S. Pat. Nos. 5,610,293, 5,637,578, 5,874,421, 5,976,498, 6,084,093, 6,180,620, 6,204,259, 6,214,817, 6,245,758, 6,395,725, and 6,525,041, each of which is incorporated herein by reference in its entirety.

The superoxide dismutase mimetics of the present invention may be represented by the following formula:

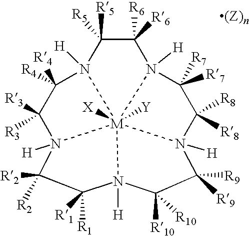

wherein

(i) R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀ are independently:

(i^{a}) hydrogen; or

(i^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(i^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(ii) optionally, one or more of R₁ or R′₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ or R′₁₀ together with the carbon atoms to which they are attached independently form a substituted or unsubstituted and saturated, partially saturated, or unsaturated cycle or heterocycle having 3 to 20 carbon atoms; and

(iii) optionally, one or more of R₁ and R′₁, R₂ and R′₂, R₃ and R′₃, R₄ and R′₄, R₅ and R′₅, R₆ and R′₆, R₇ and R′₇, R₈ and R′₈, R₉ and R′₉, and R₁₀ and R′₁₀, together with the carbon atom to which they are attached independently form a substituted or unsubstituted and saturated, partially saturated, or unsaturated-cycle or heterocycle having 3 to 20 carbon atoms; and

(iv) optionally, one or more of R₁₀ or R′₁₀ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(v) optionally, one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, together with a different one of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vi) combinations of any of (i) through (v) above.

Thus, the pentaaza-macrocyclic ligand compositions useful in the present invention can have any combinations of substituted or unsubstituted R groups, saturated, partially saturated or unsaturated cyclics, heterocyclics, nitrogen containing heterocycles, or straps as defined above.

M can be a transition metal, preferably Mn, Fe, Ni, Cu or V. X, Y and Z can independently be selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or

X, Y and Z are independently selected from the group consisting of charge-neutralizing anions which are derived from any monodentate or polydentate coordinating ligand and a ligand system and the corresponding anion thereof; or

X, Y and Z are independently attached to one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀. n is preferably an integer from 0 to 3.

Alternatively, the superoxide dismutase mimetic is represented by the formula:

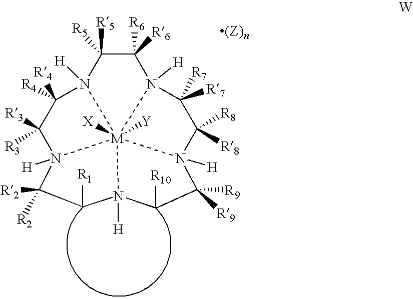

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) one or more of R₁, R₂, R′₂, R₃, R′₃, R₄, R′₄, R₅, R′₅, R₆, R′₆, R₇, R′₇, R₈, R′₈, R₉, R′₉, and R₁₀ are independently:

(ii^{a}) hydrogen; or

(ii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(ii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(iii) optionally, one or more of R₁ and R₂ or R′₁₂, R₃ or R′₃ and R₄ or R′₄, R₅, or R′₅ and R₆ or R′₆, R₇ or R′₇ and R₈ or R′₈, R₉ or R′₉ and R₁₀ together with the carbon atoms to which they are attached independently form a substituted or unsubstituted and saturated, partially saturated, or unsaturated cycle or heterocycle having 3 to 20 carbon atoms; and

(iv) optionally, one or more of R₂ and R′₂, R₃ and R′₃, R₄ and R′₄, R₅ and R′₅, R₆ and R′₆, R₇ and R′₇, R₈ and R′₈, and R₉ and R′₉ together with the carbon atom to which they are attached independently form a substituted or unsubstituted and saturated, partially saturated, or unsaturated cycle or heterocycle having: 3 to 20 carbon atoms; and

(v) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vi) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, together with a different one of R₁, R₂, R′₂, R₃, R′₃, R₄, R′₄, R₅, R′₅, R₆, R′₆, R₇, R′₇, R₈, R′₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(vii) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, may be bound to an atom of heterocycle W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) combinations of any of (i) through (vii) above.

Thus, the pentaaza-macrocyclic ligand compositions useful in the present invention can have any combinations of substituted or unsubstituted R groups, saturated, partially saturated or unsaturated cyclics, heterocyclics, nitrogen containing heterocycles, or straps as defined above, which may or may not independently connect the W loop and the pentaaza macrocycle.

M, X, Y, Z and n are preferably as defined above. W can be a substituted or unsubstituted pyridino moiety.

In another alternative, the superoxide dismutase mimetic is represented by the formula:

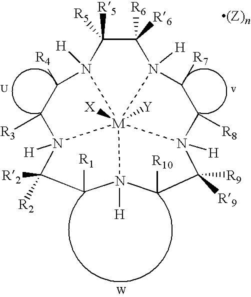

wherein

(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(ii) two sets of two adjacent carbon atoms of the macrocycle independently form substituted or unsubstituted, saturated, partially saturated or unsaturated, cycles or heterocycles U and V having 3 to 20 carbon atoms; and

(iii) R₁, R₂, R′₂, R₃, R₄, R₅, R′₅, R₆, R′₆, R₇, R₈, R₉, R′₉, and R₁₀ are independently:

(iii^{a}) hydrogen; or

(iii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or

(iii^{c}) a moiety independently selected from the group consisting of —OR₁₁, —NR₁₁R₁₂—COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and

(iv) optionally, one or more of R₁ and R₂ or R′₂, R₅ or R′₅ and R₆ or R′₆, R₉ or R′₉ and R₁₀ together with the carbon atoms to which they are attached independently form a substituted or unsubstituted and saturated, partially saturated, or unsaturated cycle or heterocycle having 3 to 20 carbon atoms; and

(v) optionally, one or more of R₂ and R′₂, R₅ and R′₅, R₆ and R′₆, and R₉ and R′₉, together with the carbon atom to which they are attached independently form a substituted or unsubstituted and saturated, partially saturated, or unsaturated cycle or heterocycle having 3 to 20 carbon atoms; and

(vi) optionally, one or more of R₂ or R′₂ and R₃, R₄ and R₅ or R′₅, R₆ or R′₆ and R₇, or R₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and

(vii) optionally, one or more of R₁, R₂, R′₂, R₃, R₄, R₅, R′₅, R₆, R′₆, R₇, R₈, R₉, R′₉, and R₁₀, together with a different one of R₁, R₂, R′₂, R₃, R₄, R₅, R′₅, R₆, R′₆, R₇, R₈, R₉, R′₉, and R₁₀, which is attached to a different carbon atom in the macrocyclic ligand may be bound to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are Independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(viii) optionally, one or more of R₁, R₂, R′₂, R₃, R₄, R₅, R′₅, R₆, R′₆, R₇, R₈, R₉, R′₉, and R₁₀, may be individually bound to an atom of heterocycles U, V and W to form a strap represented by the formula:

—(CH₂)_{I}-Q-(CH₂)_{J}—R—(CH₂)_{K}—S—(CH₂)_{L}—

wherein

I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and

(ix) combinations of any of (i) through (viii) above;

Thus, the pentaaza-macrocyclic ligand compositions useful in the present invention can have any combinations of substituted or unsubstituted R groups, saturated, partially saturated or unsaturated cyclics, heterocyclics, nitrogen containing heterocycles, or straps as defined above, which may or may not independently connect the W, U or V loops and the pentaaza macrocycle.

M, X, Y, Z and n are preferably as defined above. W can be a substituted or unsubstituted pyridino moiety. U and V can be independently saturated cycloalkyl heterocycles having 3 to 20 carbon atoms, more preferably 4 to 10 carbon atoms, still more preferably trans-cyclohexanyl fused rings. U and V can be trans-cyclohexanyl fused rings while W is a substituted pyridino moiety.

In certain embodiments, the superoxide dismutase mimetic described above the compound identified as M40403, which can be represented by the formula:

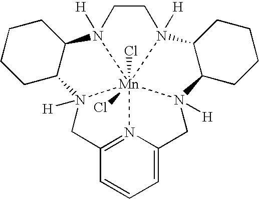

In yet another alternative, the ROS scavenger can be a peroxynitrite scavenger. The peroxynitrite scavenger can be represented by a formula selected from the group of formulas consisting of:

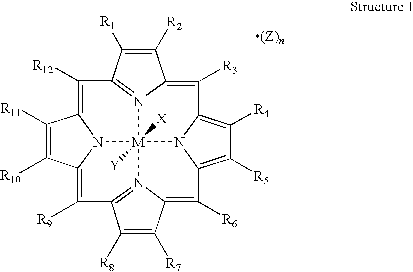

wherein R₃, R₆, R₉ and R₁₂ are independently selected from the group consisting of H, alkyl, alkenyl, CH₂, COOH, phenyl, pyridyl, and N-alkylpyridyl, such that phenyl, pyridyl and N-alkylpyridyl are:

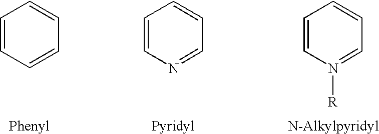

which are attached at a carbon atom; and

wherein Phenyl is optionally substituted by a substituent selected from the group consisting of a halogen, alkyl, aryl, benzyl, COOH, CONH₂, SO₃H, NO₂, NH₂, N(R)³⁺ and NHCOR′, wherein R is selected from the group consisting of hydrogen, alkyl, aryl and alkaryl, and R′ is alkyl:

wherein Pyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and

wherein N-Alkylpyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and

wherein R₁, R₂, R₄, R₅, R₇, R₈, R₁₀, or R₁₁ are independently selected from the group consisting of H, alkyl, alkenyl, carboxyalkyl, Cl, Br, F, NO₂, hydroxyalkyl, and SO₃H; and further wherein R₁ and R₂ optionally form a heterocycle having 5 to 8 carbon atoms and form a ring with the carbon atoms of the macrocycle to which they are attached;

X and Y are ligands or charge-neutralizing anions which are derived from any monodentate or polydentate coordinating ligand or ligand system or the corresponding anion thereof and are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl, amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorte, perbromate, bromate, bromite, hypobromite, tetrahalomanganate, tetrafluoroborate, hexafluorophosphate, hexafluoroanitmonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins; with the proviso that when the X and Y containing complex has a net positive charge then Z is a counter ion which is independently selected from the group consisting of X and Y, or when the X and Y containing complex has net negative charge then Z is a counter ion selected from a group consisting of alkaline and alkaline earth cations, organic cations such as alkyl or alkylaryl ammonium cations; and

M is selected from the group consisting of Mn, Fe, Ni and V; and

n is an integer from 0 to 4.

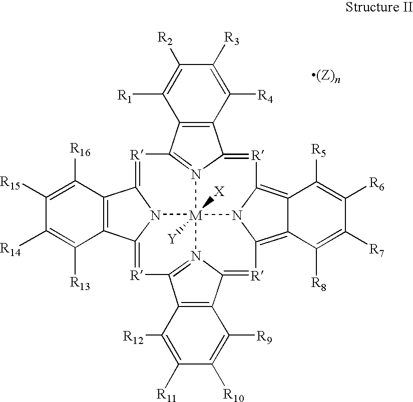

wherein R′ is CH or N;

R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of H, SO₃H, COOH, NO₂, NH₂, and N-alkylamino; and

X, Y, Z, M and n are as defined above;

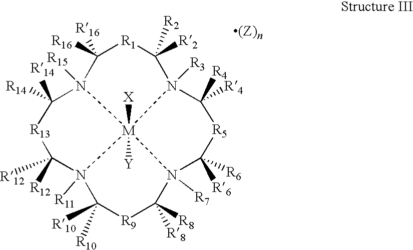

A

wherein R₁, R₅, R₉, and R₁₃ are independently selected from the group consisting of a direct bond and CH₂;

R₂, R′₂, R₄, R′₄, R₆, R′₆, R₈, R′₈, R₁₀, R′₁₀, R₁₂, R′₁₂, R₁₄, R′₁₄, R₁₆, and R′₁₆ are independently selected from the group consisting of H and alkyl;

R₃, R₇, R₁₁, and R₁₅ are independently selected from the group consisting of H and alkyl; and

X, Y, Z, M and n are as defined above;

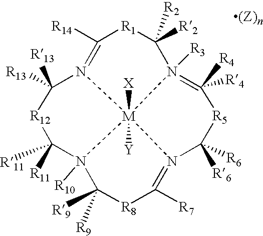

B

wherein R₁, R₅, R₈, and R₁₂ are independently selected from the group consisting of a direct bond and CH₂;

R₂, R′₂, R₄, R′₄, R₆, R′₆, R₇, R₉, R′₉, R₁₁, R′₁₁, R¹³, R′₁₃, and R₁₄ are independently selected from the group consisting of H and alkyl;

R₃ and R₁₀ are independently selected from the group consisting of H and alkyl; and

X, Y, Z, M and n are as defined above;

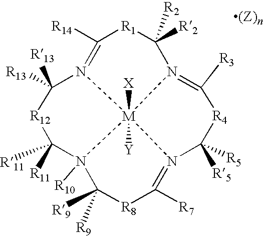

C

wherein R₁, R₄, R₈, and R₁₂ are independently selected from the group consisting of a direct bond and CH₂;

R₂, R′₂, R₃, R₅, R′₅, R₇, R₉, R′₉, R₁₁, R′₁₁, R₁₃, R′₁₃ and R₁₄ are independently selected from the group consisting of H and alkyl;

R₁₀ is H or alkyl; and

X, Y, Z, M and n are as defined above;

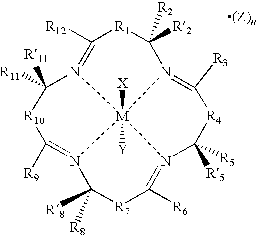

D

wherein R₁, R₄, R₇ and R₁₀ are independently selected from the group consisting of a direct bond and CH₂;

R₂, R′₂, R₃, R₅, R′₅, R₆, R₈, R′₈, R₉, R₁₁, R′₁₁ and R₁₂ are independently selected from the group consisting of H and alkyl; and

X, Y, Z, M and n are as defined above;

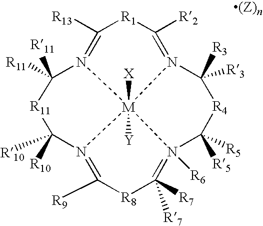

E

wherein R₁, R₄, R₈ and R₁₁ are independently selected from the group consisting of a direct bond and CH₂;

R₂, R₃, R′₃, R₅, R′₅, R₇, R′₇, R₉, R₁₀, R′₁₀, R₁₂, R′₁₂ and R₁₃ are independently selected from the group consisting of H and alkyl;

R₆ is hydrogen or alkyl; and

X, Y, Z, M and n are as defined above;

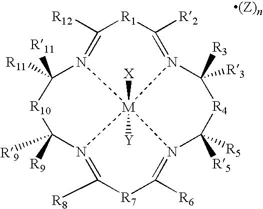

F

wherein R₁, R₄, R₇ and R₁₀ are independently selected from the group consisting of H and alkyl;

R₂, R₃, R′₃, R₅, R′₅, R₆, R₈, R₉, R′₉, R₁₁, R′₁₁ and R₁₂ are independently selected from the group consisting of H and alkyl; and

X, Y, Z, M and n are as defined above;

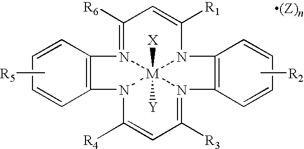

G

wherein R₁, R₃, R₄, and R₆ are independently selected from the group consisting of H and alkyl;

R₂ and R₅ are independently selected from the group consisting of H, alkyl, SO₃H, NO₂, NH₂, halogen, COOH and N(R)³⁺ wherein R is as defined above; and

X, Y, Z, M and n are as defined above;

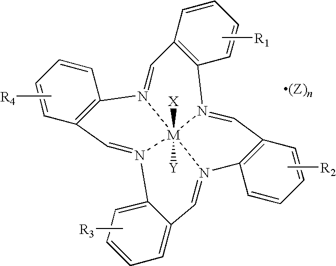

H

wherein R₁, R₂, R₃, and R₄ are independently selected from the group consisting of H, alkyl, SO₃H, NO₂, NH₂, halogen, COOH and N(R)³⁺ wherein R is as defined above; and

X, Y, Z, M and n are as defined above; and

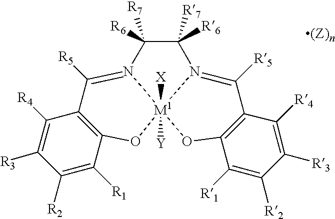

wherein R₁, R′₁, R₂, R′₂, R₃, R′₃, R₄, R′₄, R₅, R′₅, R₆, R′₆, R₇ and R′₇ are independently selected from the group consisting of H, alkyl, alkoxy, NO₂, aryl, halogen, NH₂ and SO₃H, further wherein R₆, R′₆, R₇ and R′₇ together with one other of R₆, R′₆, R₇ and R′₇ optionally form a heterocycle having 5 to 8 carbon atoms and form a ring with the carbon atoms of the macrocycle to which they are attached;

M¹ is selected from the group consisting of Fe, Ni or V; and

X, Y, Z and n are as defined above.

The present invention can involve administration of methotrexate and a ROS scavenger, in particular, M40403 or a suitable derivative or analog thereof as described above, to a patient in need thereof. In such combination treatment, either or both of the methotrexate or the ROS scavenger are administered at a relatively low dose compared to that producing maximal beneficial effects on the inflammatory disease. Such low doses by themselves would not be expected to produce a substantial remedial effect on inflammation, however, when given in combination, a substantial beneficial effect is seen as a result of a synergistic interaction of the methotrexate and the ROS scavenger, in particular, M40403. Additive and synergistic combinations can be determined by the methods provided in the Examples below.

Administration of the methotrexate and ROS scavenger, in particular M40403, can be by the same or different routes of administration. Administration can also be together in one composition or in separate compositions. In addition administration can be substantially at the same time or at different times and on a different schedule of administration. For example, it is possible to administer methotrexate on a schedule of once or twice a week and to administer the ROS scavenger on a schedule of once, twice or three times a day as described more fully below.

Administration of methotrexate and the ROS scavenger can be by any suitable route of administration such as, for example, oral, buccal, sublingual, intranasal, inhalation, rectal, intravaginal, transdermal, intradermal, subcutaneous, intramuscular, intraperitoneal, intravenous, intraarterial, intrasternal, intrathecal and the like.

Pharmaceutically acceptable formulations for parenteral or nonparenteral drug delivery are known in the art such as, for example, are set forth in Remington's Pharmaceutical Sciences, 18th Edition, Mack Publishing (1990). Pharmaceutical compositions can be formulated to be compatible with the intended route of administration. Solutions or suspensions used for parenteral, intradermal or subcutaneous application can include: a sterile diluent, such as water for injection, saline solution, fixed oils, polyethylene glycols, glycerine, propylene glycol or other synthetic solvents, antibacterial agents, such as benzyl alcohol or methyl parabens; antioxidants, such as ascorbic acid or sodium bisulfite; chelating agents, such as ethylenediaminetetraacetic acid (EDTA); buffers such as acetates, citrates or phosphates, and agents for the adjustment of tonicity, such as sodium chloride or dextrose. Suitable carriers include physiological saline, bacteriostatic water, Cremophor® EL (BASF, Parsippany, N.J.) or phosphate buffered saline (PBS). The compositions can be stable during manufacture and storage and preserved against contamination from microorganisms, such as bacteria and fungi. Proper fluidity can be maintained, for example, by using a coating such as lecithin; by maintaining the required particle size in the case of dispersion, and by using surfactants. Various antibacterial and antifungal agents, such as parabens, chlorobutanol, phenol, ascorbic acid, and thimerosal, can control microorganism contamination. Isotonic agents, such as sugars, polyalcohols such as mannitol, sorbitol, and sodium chloride can be included in the composition. Compositions that delay absorption can be prepared by including such agents as aluminum monostearate and gelatin.

Sterile injectable solutions can be prepared by incorporating the active compound (e.g., an SCMP) in an appropriate solvent with one or more ingredient, followed by sterilization. Generally, dispersions are prepared by incorporating the active compound into a sterile vehicle that contains a basic dispersion medium and any other required ingredients. Sterile powders for the preparation of sterile injectable solutions include vacuum- and freeze-drying that yield a powder containing the active ingredient and any desired ingredient from a sterile solution. The concentration of active drug, i.e. either or both of methotrexate and the ROS scavenger can be from about 0.1% to about 90% by weight, from about 5% to about 20% by weight, from about 5% to about 17% by weight, from about 8% to about 14% by weight or, in certain embodiments, about 10% by weight.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring. The concentration of active drug, i.e. either or both of methotrexate and the ROS scavenger can be from about 0.1% to about 99% by weight, from about 5% to about 95% by weight, from about 10% to about 90% by weight, from about 15% to about 85% by weight, from about 20% to about 80% by weight, from about 25% to about 75% by weight, from about 30% to about 70% by weight, from about 35% to about 64% by weight or from about 40% to about 60% by weight.

Administration by inhalation, can be by aerosol spray from a nebulizer or a pressurized container that contains a suitable propellant, e.g., a gas such as carbon dioxide.

Systemic administration can also be transmucosal or transdermal. For transmucosal or transdermal administration, penetrants that can permeate the target barrier(s) are selected. Transmucosal penetrants include detergents, bile salts and fusidic acid derivatives. Nasal sprays or suppositories can be used for transmucosal administration. For transdermal administration, the active compounds are formulated into ointments, salves, gels or creams.

The compounds can also be prepared as suppositories (with bases such as cocoa butter and other glycerides) or retention enemas for rectal delivery.

In various embodiments, the active compounds can be prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid (Alza Corporation; Mountain View, Calif. and Nova Pharmaceuticals, Inc.; Lake Elsinore, Calif.). Liposomal suspensions can also be used as pharmaceutically acceptable carriers (Eppstein, 1985).

Oral formulations or parenteral compositions in unit dosage form can be created to facilitate administration and dosage uniformity. Unit dosage form refers to physically discrete units suited as single doses for a subject to be treated, containing a therapeutically effective quantity of active compound in association with the required pharmaceutical carrier. The specification for unit dosage forms are dictated by, and directly dependent on, the unique characteristics of the active compound and the particular desired therapeutic effect, and the inherent limitations of compounding the active compound.

Methotrexate can be given by any suitable route, and generally, by oral administration or by intramuscular or subcutaneous injection. Doses of methotrexate can be from about 0.01 mg up to about 100 mg or from about 0.001 mg/kg up to about 1 mg/kg body weight. Doses showing therapeutic response in human patients when administered alone, can be 7.5 mg, 15 mg, 20 mg 30 mg or 50 mg administered once a week (see for example, Jones et al., Am. Fam. Physician 62:1607-14, 2000; Perhala et al., Comprehensive Therapy 17:51-60, 1991). Low doses of methotrexate of the present invention include doses less than about 7.5 mg or less than about 0.1 mg/kg including about 0.07 mg, about 0.1 mg, about 0.15 mg, about 0.35 mg, about 0.5 mg, about 0.6 mg, about 0.7 mg, about 1 mg, about 1.5 mg, about 3.5 mg about 6 mg or about 0.001 mg/kg, about 0.0015 mg/kg, about 0.002 mg/kg, about 0.005 mg/kg, about 0.008 mg/kg, about 0.01 mg/kg, about 0.015 mg/kg, about 0.02 mg/kg, about 0.05 mg/kg, or about 0.08 mg/kg body weight.

Administration of methotrexate can be on a weekly dosing schedule in a single dose or in divided doses given two, three or four times during a 24 hour period as is typical for therapeutically effective doses administered in absence of a ROS scavenger. The low doses of methotrexate of the present invention are however, believed to have a diminished propensity for producing side effects such that administration can be on a more frequent dosing schedule, such as, for example twice a week, once a day, twice a day, three times a day or four times a day.

A typical dose of the ROS scavenger can be from about 0.1 mg up to about 1000 mg or from about 0.001 up to about 10 mg/kg body weight. Doses of 5 mg/kg and 10 mg/kg administered intraperitoneally have shown to produce beneficial effects in rats treated with type II collagen to induced arthritis (Salvemini et al., Arthritis & Rheumatism, 44:2909-2921, 2001). A dose of 2 mg/kg produced less of an effect that was nevertheless significantly different from that in placebo animals. Low doses of a ROS scavenger can be doses of less than about 5 mg/kg or doses equal to or less than about 2 mg/kg body weight, in particular, a dose of about 0.1 mg, about 0.2 mg, about 0.5 mg, about 0.8 mg, about 1 mg, about 2 mg, about 5 mg, about 8 mg, about 10 mg, about 20 mg, about 50 mg, about 80 mg, about 100 mg or about 200 mg or about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, about 1 mg/kg, about 2 mg/kg, about 3 mg/kg or about 4 mg/kg body weight.

Total daily doses of the ROS scavenger can be administered in single or divided doses and in amounts such as, for example, from about 1 to about 2 mg/kg body weight daily and more usually about 0.05 to 1 mg/kg. Dosage unit compositions may contain such amounts of submultiples thereof to make up the total dose. However, one skilled in the art will recognize that the total dosage will vary on the particular composition the particular ROS scavenger administered.

Individuals receiving treatment are, typically, human patients, however, patients receiving treatment can also be animal including companion animal such as dogs and cats, farm animal such as cows, horses, swine as well as birds and exotic animal such as zoo animals.

The amount of active-ingredients that may be combined with the carrier materials to produce a single dosage form can vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention can selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed can, therefore, can deviate from the preferred dosage regimen set forth above.

In various embodiments, the present invention can also involve kits. Such kits can include pharmaceutical compositions and in addition in certain embodiments, instructions for administration. When supplied as a kit, the different components of the composition can be packaged in separate containers and admixed immediately before use. Such packaging of the components separately can, in certain instances, permit long-term storage without losing activity of the components. In addition, if more than one route of administration is intended or more than one schedule for administration is, intended, the different components can be packaged separately and not mixed prior to use. In various embodiments, the different components can be packaged in one composition for administration together.

Kits may also include reagents in separate containers such as, for example sterile water or saline to be added to a lyophilized active component packaged separately. For example, sealed glass ampules may contain lyophilized ROS scavenger or methotrexate and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, etc. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

The compositions and methods of the present invention are effective in treating diseases and conditions involving inflammation. Such diseases and conditions can include, for example, rheumatoid arthritis, psoriasis, inflammatory bowel disease including ulcerative colitis and Crohn's disease, corticosteroid-dependent asthma, multiple sclerosis, lupus erythematosus and the like.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Methotrexate and ROS Scavenger Combinations

Animals

Male Lewis rats (160-180 g; Charles River; Milan; Italy) were housed in a controlled environment and provided with standard rodent chow and water. Animal care was in compliance with Italian regulations on protection of animals used for experimental and other scientific purposes (D.M. 116192) as well as with the EEC regulations (O.J. of E.C. L 358/1 Dec. 18, 1986).

Experimental Protocol

Animals were randomly divided into sixth groups (n=10 for each group); (1) Sham group receiving intraperitoneally 26 mM sodium bicarbonate buffer at pH 8.1-8.3 which constituted the vehicle for M40403; (2) CIA group which constituted rats subjected to collagen induced arthritis as described below; (3) CIA+M40403 (2 mg/kg) group in which rats subjected to collagen-induced arthritis received M40403 at 2 mg/kg i.p. every 24 h, starting from day 25; (4) CIA+MET (0.015 mg/kg) group in which rats subjected to collagen-induced arthritis received orally, methotrexate at 0.015 mg/kg starting from day 25; (5) CIA+MET (0.15 mg/kg) group in which rats subjected to collagen-induced arthritis received orally, methotrexate at 0.15 mg/kg starting from day 25; (6)

CIA+MET+M40403 group in which rats subjected to collagen-induced arthritis received methotrexate (0.015 mg/kg; orally) and with M4043 (2 mg/kg, i.p.) starting from day 25.

Induction of Collagen-Induced Arthritis

Collagen-induced arthritis was produced as follows. Bovine type II collagen was dissolved in 0.01 M acetic acid at a concentration of 2 mg/ml by stirring overnight at 4° C. Dissolved collagen was frozen at −70° C. until use. Complete Freund's adjuvant was prepared by the addition of Mycobacterium tuberculosis H37Ra at a concentration of 2 mg/ml. Before injection, the collagen was emulsified with an equal volume of complete Freund's adjuvant. Collagen-induced arthritis was elicited as previously described (Salvemini et al Arthritis & rheumatism 44:2909-2921, 2001). On day 1, Lewis rats were injected intradermally at the base of the tail with 100 μl of the emulsion (containing 100 μg of bovine type II collagen). On day 21, a second injection of type II collagen in complete Freund's adjuvant was administered.

Clinical Assessment of Collagen-Induced Arthritis.

Rats were evaluated daily for arthritis by using a macroscopic scoring system as follows: 0=no signs of arthritis; 1=swelling and/or redness of the paw or one digit; 2=two joints involved; 3=more than two joints involved; and 4=severe arthritis of the entire paw and digits. Arthritic index for each rats was calculated by adding the four scores of individual paws. Clinical severity was also determined by quantitating the change in the paw volume using plethysmometry (model 7140; Ugo Basile).

Histological Assessment of Joint Injury

At day 35, animals were sacrificed while they were under anesthesia, and paws and knees were removed and fixed for histological examination. The examination was performed by an investigator blinded for the treatment regime. The following morphological criteria were considered: score 0, no damage; score 1, oedema; score 2, inflammatory cell presence; score 3, bone resorption.

Histological Examination

Biopsies of paws and knees obtained at 35 days were fixed for 1 week in buffered formaldehyde solution (10% in phosphate buffered saline) at room temperature, dehydrated by graded ethanol and embedded in Paraplast (Sherwood Medical, Mahwah, N.J.). The paws were trimmed, placed in decalcifying solution for 24 h, embedded in paraffin, sectioned at 5 g. Tissue sections were deparaffinized with xylene, stained with trichromic Van Gieson and studied using light microscopy (Dialux 22 Leitz).

Radiography

Rats were anaesthetised with sodium pentobarbital (45 mg/kg, i.p.) and placed on a radiographic box at a distance of 90 cm from the x-ray source. Radiographic analysis of normal and arthritic rat hind paws was performed by x-ray machine (Philips X12 Germany) with a 40 kW exposition for 0.01 sec. An investigator blinded for the treatment regime performed radiograph score. The following radiograph criteria were considered: score 0, no bone damage; score 1, tissue swelling and oedema; score 2 joint erosion; 3, bone erosion and osteophyte formation.

Measurement of Cytokines

TNF-α and IL-1β levels were evaluated in plasma at 35 days after the induction of arthritis. The assay was carried out by using a calorimetric, commercial kit (Calbaiochem-Novabiochem Corporation, USA). The ELISA has a lower detection limit of 5 pg/ml.

Materials

Unless otherwise stated, all compounds of this Example were obtained from Sigma-Aldrich Company Ltd. (Poole, Dorset, UK). Thiopentone sodium (Intraval Sodium®) was obtained from Rhône Mérieux Ltd. (Harlow, Essex, UK). All other chemicals were of the highest commercial grade available. All stock solutions were prepared in nonpyrogenic saline (0.9% NaCl; Baxter Healthcare Ltd., Thetford, Norfolk, UK).

Data Analysis

All values in the figures and text are expressed as mean standard error (s.e.m.) of the mean of n observations. For the in vivo studies n represents the number of animals studied. In the experiments involving histology or immunohistochemistry, the figures shown are representative of at least three experiments performed on different experimental days. Data sets were examined by one- and two-way analysis of variance, and individual group means were then compared with Student's unpaired t test. For the arthritis studies, Mann-Whitney U test (two-tailed, independent) was used to compare medians of the arthritic indice. Values in for the in vitro studies are presented as incidences (%), or medians. A p-value less than 0.05 was considered significant

Results

Effect of Combination therapy in the Development of Collagen-Induced Arthritis

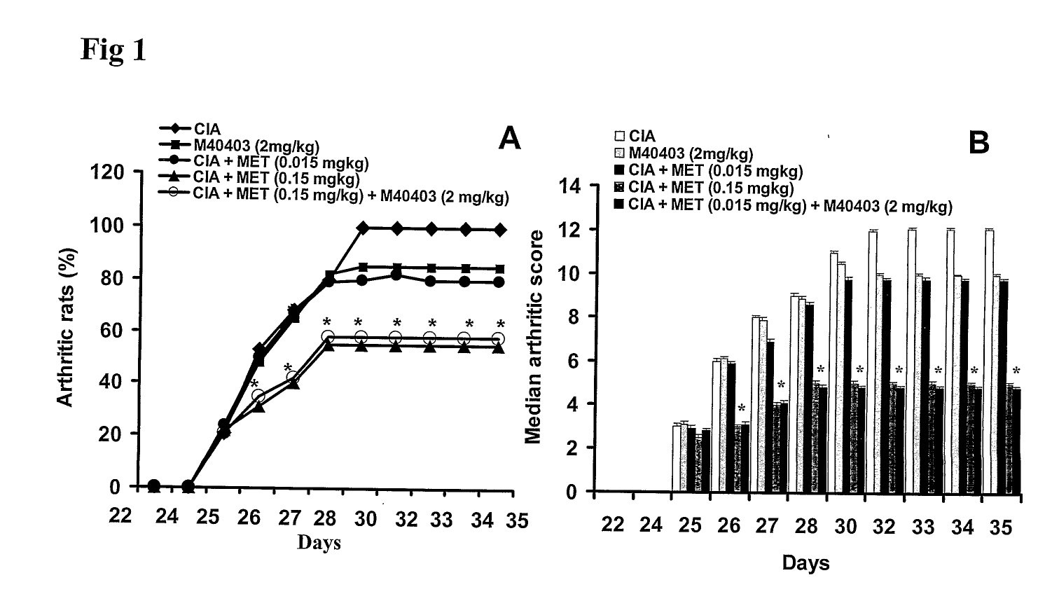

Collagen-induced arthritis developed rapidly in rats immunised with type II collagen and clinical signs (periarticular erythema and oedema) of the disease (FIG. 1A) first appeared in the hind paws between 24 to 26 days post-challenge. Furthermore, a 100% incidence of collagen-induced arthritis was observed by day 28 in rats immunized with type II collagen. M40403 (2 mg/kg; Metaphore Pharmaceuticals, Inc., St. Louis, Mo.) or methotrexate (0.015 mg/kg) treatment alone did not significantly altered the development of collagen-induced arthritis although a small, non-significant attenuation was observed. Methotrexate at a concentration of 0.15 mg/kg significantly produced a significant reduction in the percent of arthritic rats. The combination therapy with M40403 (2 mg/kg) and methotrexate (0.015 mg/kg) also significantly reduced the development of the inflammatory process (FIG. 1A) and no significant difference was found between the effects of the combination therapy and that of methotrexate at 0.15 mg/kg. Neither the clinical signs nor histopathological features of collagen-induced arthritis were observed in rat fore paws during the evaluation period.

Hind paw erythema and swelling increased in frequency and severity in a time-dependent mode with maximum arthritis indices of approximately 13 observed between 28 to 35 days post-immunization (FIG. 1B). Neither M40403 (2 mg/kg) nor methotrexate. (0.015 mg/kg) treatment alone attenuated the arthritis index (FIG. 1B). Methotrexate (0.15 mg/kg) exerted a significant suppression (P<0.01) of the arthritis index between days 26 and 35 days after immunization (FIG. 1B). The combination therapy with M40403 (2 mg/kg) and methotrexate (0.015 mg/kg) significantly reduced the arthritis index (FIG. 1B). No significant difference was found between the combination therapy and the higher dose of MET (0.15 mg/kg).

There was no macroscopic evidence of either hind paw erythema or oedema in the sham rats (data not shown).

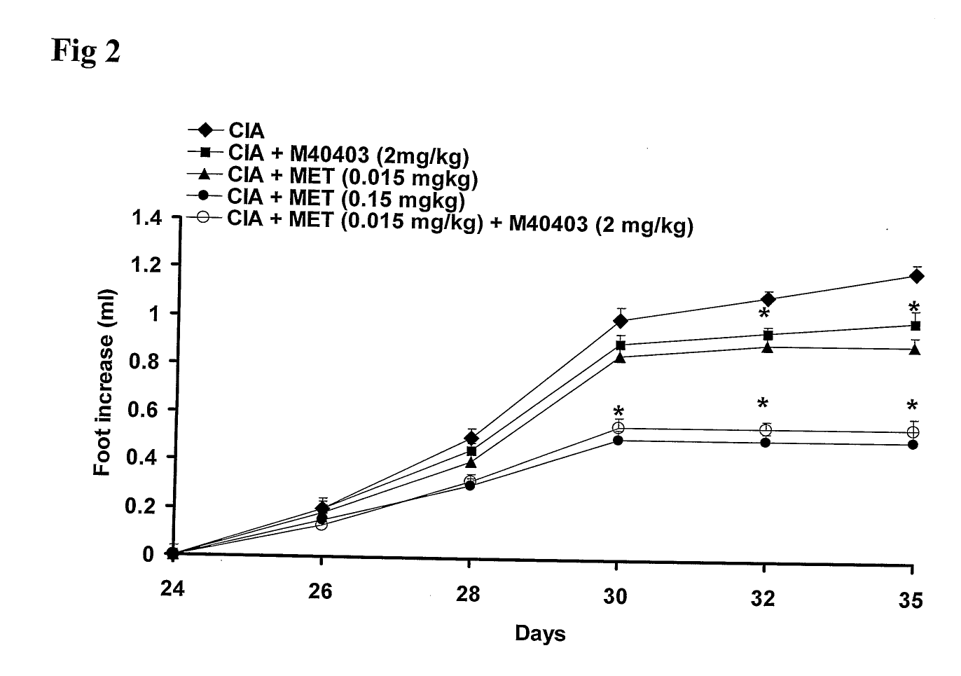

The data in FIG. 2 demonstrate a time-dependent increase in hind paw (each value represents the mean values of both hind paws) volume (ml) in rats immunized with type-II collagen. Maximum paw volume occurred by day 35 in the rats immunized with type-II collagen.

M40403 (2 mg/kg) or methotrexate (0.015 mg/kg) treatment attenuated the paw edema from day 30 to 35. Treatment with methotrexate (0.15 mg/kg) alone or with the combination therapy (methotrexate, 0.015 mg/kg+M40403, 2 mg/kg) exhibited a continuously significant (P<0.001) suppression of hind paw swelling from day 26 to 35 post-immunization, achieving a maximal response of 70% from day 28 to 35 (FIG. 2). No significant difference was found between the effect of the higher dose of methotrexate and that of the combination therapy. No increases in hind paw volume over time was observed with normal control (data not shown).

Effects of combination therapy on Histopathology and Radiographic analysis of Collagen Induced Arthritis

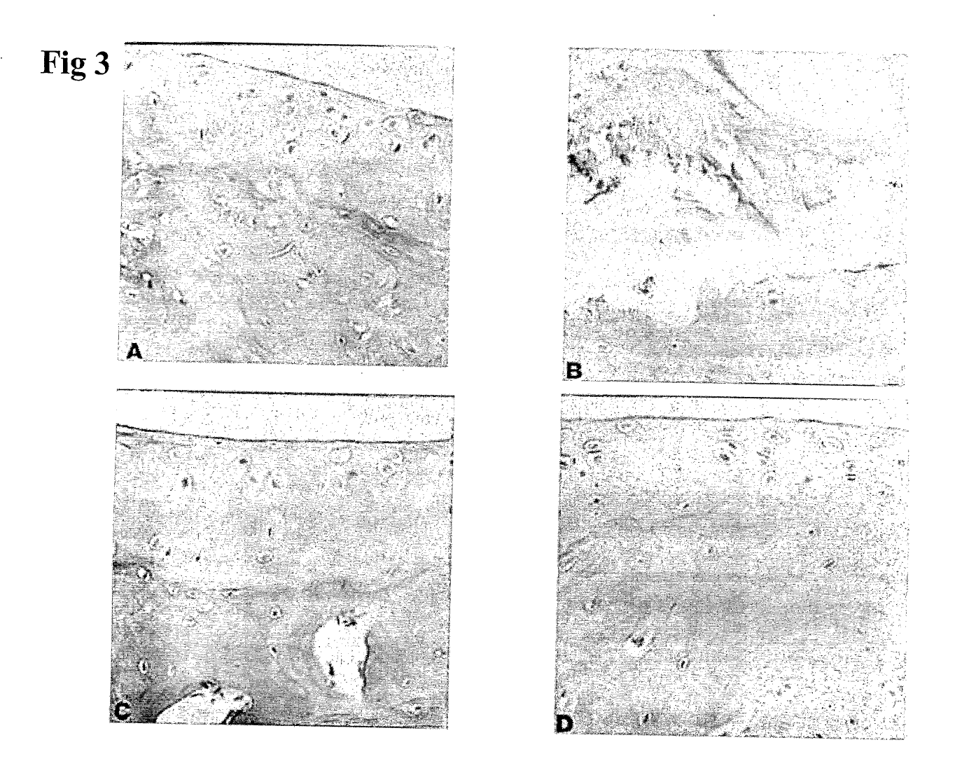

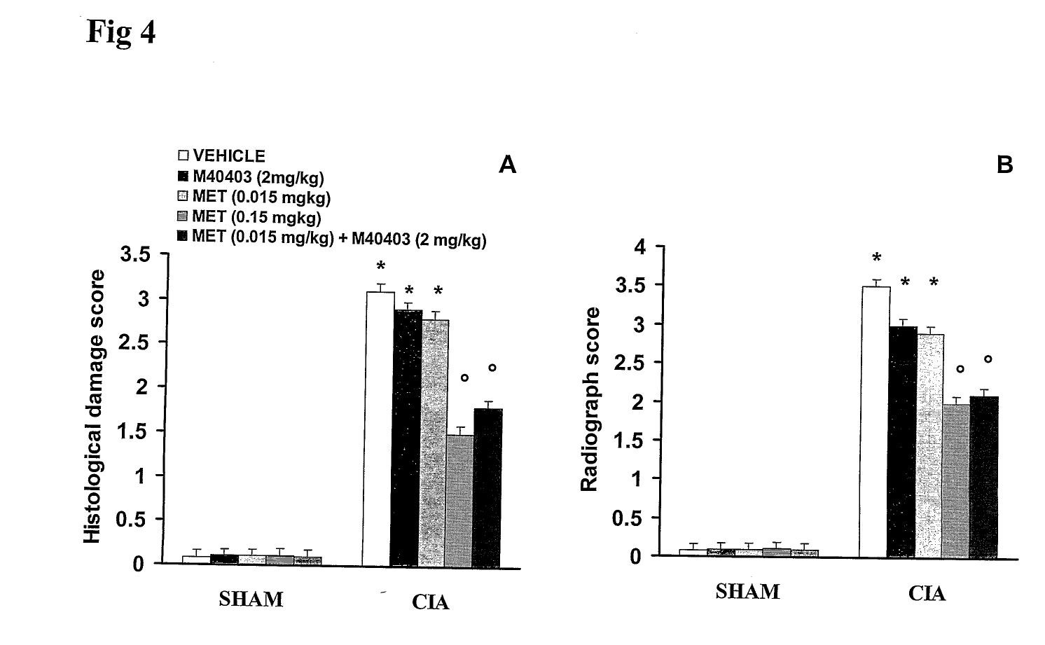

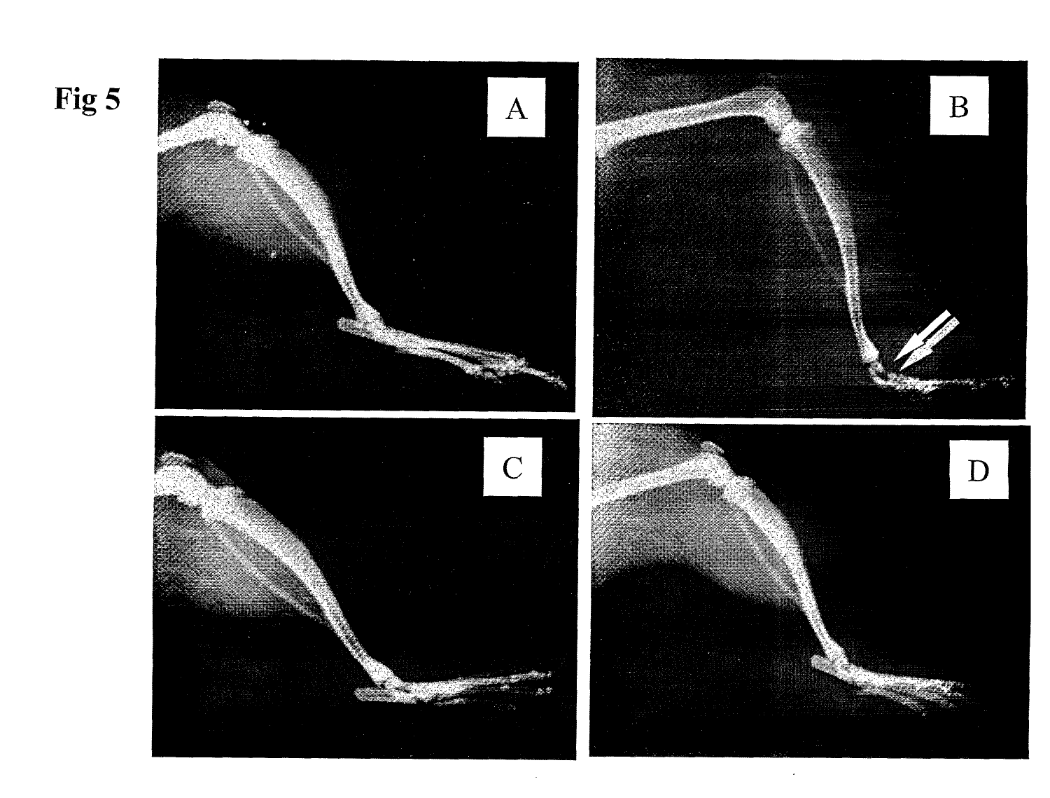

At day 35, histological evaluation of the paws in the vehicle-treated arthritic animals revealed signs of severe suppurative arthritis, with bone resorption (FIG. 3B). In addition, severe or moderate necrosis, hyperplasia and sloughing of the synovium could be seen; together with the extension of the inflammation into the adjacent musculature with fibrosis and increased mucous production (FIG. 4A for damage score). In the animals which received methotrexate (0.15 mg/kg; FIG. 3C) or the combination therapy (methotrexate, 0.015 mg/kg+M40403, 2 mg/kg; FIG. 3D) the bone erosion as well as the degree of arthritis were significantly reduced (FIG. 4A for damage score). A radiographic examination of hind paws from rats 35 days post immunization with type-II collagen revealed bone matrix resorption (FIG. 5B), osteophyte formation at the joint margin and soft tissue swelling in the tibiotarsal joint (FIG. 4B for radiographic score). There was no evidence of pathology in sham rats (FIGS. 3A and 5A).

Methogtrexate (0.15 mg/kg; FIG. 5C) or the combination therapy (methotrexate, 0.015 mg/kg+M40403, 2 mg/kg; FIG. 5D) markedly protect from bone resorption. No significant protection was found in the animal treated with M40403 2 mg/kg or with methotrexate at a dose of 0.015 mg/kg (FIG. 4).

Effect of Combination Therapy on Cytokine Production

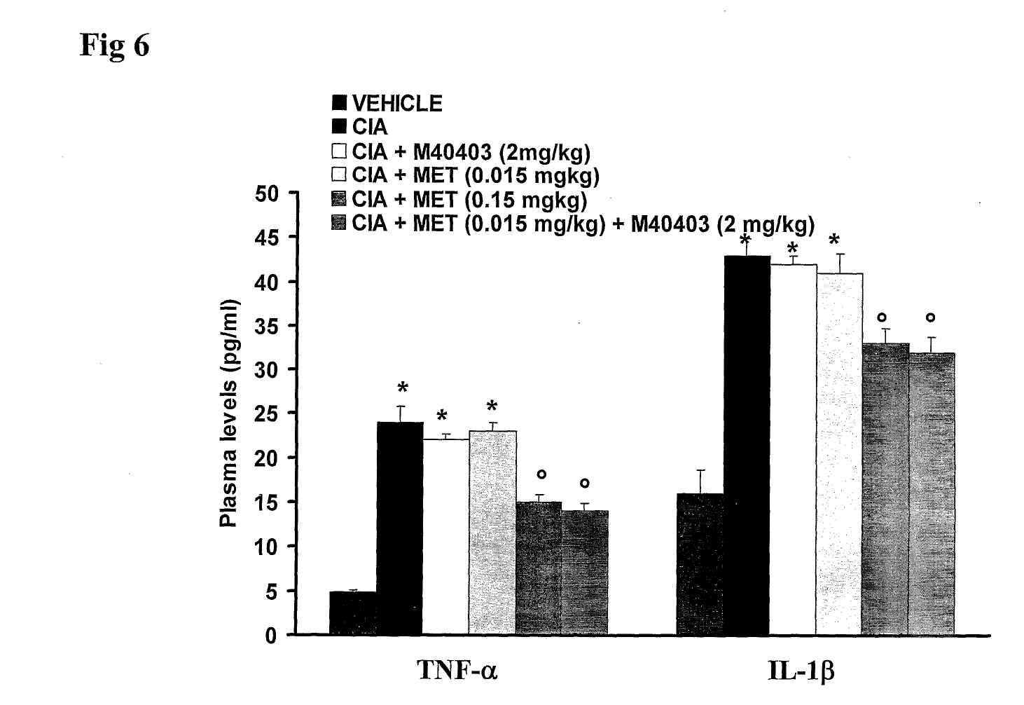

At day 35, the levels of TNF-α and IL-1β were significantly elevated in the plasma from CIA-treated rats (FIG. 6). In contrast, the levels of these cytokines were significantly lower in rats which received methotrexate (0.15 mg/kg) or the combination therapy (methotrexate, 0.015 mg/kg+M40403, 2 mg/kg) (FIG. 6). No significant effect was found the in the animal treated with M40403, 2 mg/kg or with methotrexate 0.015 mg/kg. No significant cytokines increased was observed in the plasma of sham rats.

Effect of Combination Therapy on Body Weight Gain

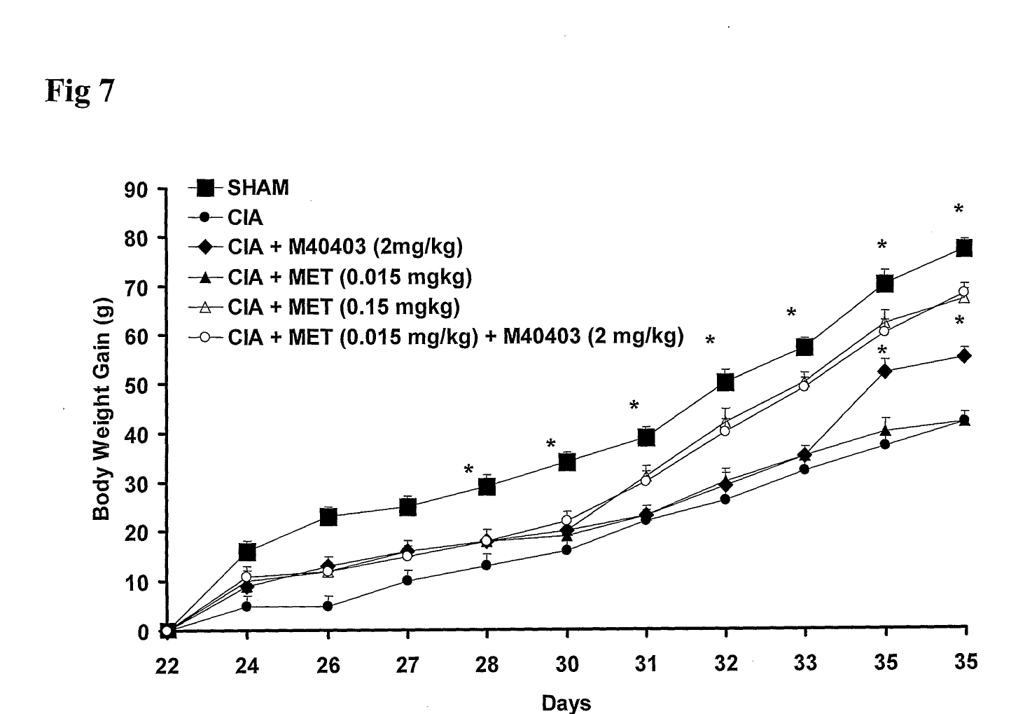

The rate of change and the absolute gain in body weight were comparable in sham Lewis rats and rats immunized with type-II collagen for the first week (FIG. 7). Beginning on day 25, the collagen-challenged rats gained significantly less weight than the normal rats, and this trend continued through day 35. Methotrexate (0.15 mg/kg) or the combination therapy (methotrexate, 0.015 mg/kg+M40403, 2 mg/kg) positively affected the weight gain of immunized rats (FIG. 7). Treatment with methotrexate (0.015 mg/kg) or with M40403 (2 mg/kg) significantly attenuated the loss in body weight.

### Example 2

### Determining Additive and Synergistic Effects

Studies employing combinations of drugs may be analyzed for additive or synergistic interactions by isobolographic analysis as described by Tallarida (Tallarida et al., Life Sciences, 45:947-61, 1987) and employed by others (Ossipov et al., J. Pharmacol. Exp. Ther., 255:1107-1116, 1990; Porreca et al., Euro. J. Pharm., 179: 463-468, 1990) by means of a customized Visual Basic computer program (Ossipov, personal communication). Log dose-response curves for each component administered alone may be established and the A₅₀ (95% C.L.) may be calculated.

Using these methods, the amount of synergy of a combination of methotrexate and a ROS scavenger can be determined. The preferred combinations of the present invention treat inflammation using a smaller dose of methotrexate when compared to administering the methotrexate alone. In other words, a preferred combination will result, for example, in the same amount of pain relief after administering 50 mg of methotrexate in combination with 50 mg of a ROS scavenger as would normally result from administering 500 mg of methotrexate alone or 500 mg of a ROS scavenger alone.

Conversely, the preferred combinations of the present invention treat inflammation to a greater extent when compared to treating inflammation with methotrexate alone or a ROS scavenger alone. In other words, a preferred combination will result, for example, in an equivalent amount of inflammation relief after administering 500 mg of methotrexate in combination with 50 mg of ROS scavenger as would normally result from administering 1,000 mg of the methotrexate or 1,000 mg of a ROS scavenger alone.

Thus, preferred combinations result in additive or synergistic antiinflammatory effects allowing the individual methotrexate or ROS scavenger component of a methotrexate and ROS scavenger combination to be administered at a dosage which contains less than 50% of the methotrexate or ROS scavenger to achieve the same antiinflammatory effect when compared to administering the methotrexate or ROS scavenger alone. More preferably, the methotrexate and ROS scavenger combination may be administered in a dosage that contains less than 25% of the individual methotrexate or ROS scavenger component to achieve the same antiinflammatory effect. Still more preferably, the methotrexate and ROS scavenger combination may be administered in a dosage that contains less than 10% of the individual methotrexate or ROS scavenger component to achieve the same antiinflammatory effect. And still more preferably, the methotrexate and ROS scavenger combination may be administered in a dosage that contains less than 1% of the individual methotrexate or ROS scavenger component to achieve the same antiinflammatory effect.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method for treating an inflammatory disease, the method comprising administering to a patient in need thereof, methotrexate and a ROS scavenger in a pharmaceutically acceptable formulation.

2. A method according to claim 1, wherein the ROS scavenger is a superoxide dismutase mimetic.

3. A method according to claim 2, wherein the superoxide dismutase mimetic is represented by the formula: 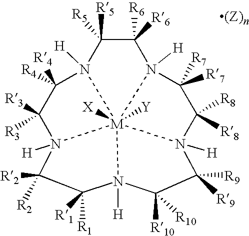
wherein
(i) R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀ are independently:
(i^{a}) hydrogen; or
(i^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(i^{c}) a moiety independently selected from the group consisting of —OR₁₁, —NR₁₁R₁₂, —COR₁₁, —CO₂R₁₁, —CONR₁₁R₁₂, —SR₁₁—SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(ii) optionally, one or more of R₁ or R′₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ or R′₁₀ together with the carbon atoms to which they are attached independently form a substituted or unsubstituted and saturated, partially saturated, or unsaturated cycle or heterocycle having 3 to 20 carbon atoms; and
(iii) optionally, one or more of R₁ and R′₁, R₂ and R′₂, R₃ and R′₃, R₄ and R′₄, R₅ and R′₅, R₆ and R′₆, R₇ and R′₇, R′₈ and R′₈, R₉ and R′₉, and R₁₀ and R′₁₀, together with the carbon atom to which they are attached independently form a substituted or unsubstituted and saturated, partially saturated, or unsaturated cycle or heterocycle having 3 to 20 carbon atoms; and
(iv) optionally, one or more of R₁₀ or R′₁₀ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, together with a different one of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, arylithiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀; and
n is an integer from 0 to 3.

4. A method according to claim 3, wherein M is selected from the group consisting of Mn, Fe, Ni, Cu and V.

5. A method according to claim 2, wherein the superoxide dismutase mimetic is represented by the formula: 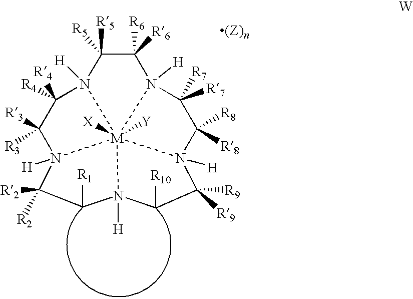
wherein
(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(ii) one or more of R₁, R₂, R′₂, R₃, R′₃, R₄, R′₄, R₅, R′₅, R₆, R′₆, R₇, R′₇, R₈, R′₈, R₉, R′₉, and R₁₀ are independently:
(ii^{a}) hydrogen; or
(ii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(iii) optionally, one or more of R₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ together with the carbon atoms to which they are attached independently form a substituted or unsubstituted and saturated, partially saturated, or unsaturated cycle or heterocycle having 3 to 20 carbon atoms; and
(iv) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted and saturated, partially saturated, or unsaturated cycle or heterocycle having 3 to 20 carbon atoms; and
(v) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(vi) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, together with a different one of R₁, R₂, R′₂, R₃, R′₃, R₄, R′₄, R₅, R′₅, R₆, R′₆, R₇, R′₇, R₈, R′₈, R₉, R′₉, and R₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vii) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, may be bound to an atom of heterocycle W to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(viii) combinations of any of (i) through (vii) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrite, aryl nitrite, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R₂, R′₂, R₃, R′₃, R₄, R′₄, R₅, R′₅, R₆, R′₆, R₇, R′₇, R₈, R′₈, R₉, R′₉, and R₁₀; and
n is an integer from 0 to 3.

6. A method according to claim 5, wherein M is selected from the group consisting of Mn, Fe, Ni, Cu and V.

7. A method according to claim 5, wherein W is a substituted or unsubstituted pyridino moiety.

8. A method according to claim 2, wherein the superoxide dismutase mimetic is represented by the formula: 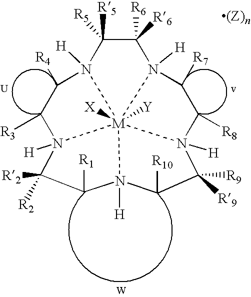
wherein
(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(ii) two sets of two adjacent carbon atoms of the macrocycle independently form substituted or unsubstituted, saturated, partially saturated or unsaturated, cycles or heterocycles U and V having 3 to 20 carbon atoms; and
(iii) R₁, R₂, R′₂, R₃, R₄, R₅, R′₅, R₆, R′₆, R₇, R₈, R₉, R′₉, and R₁₀ are independently:
(iii^{a}) hydrogen; or
(iii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(iii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(iv) optionally, one or more of R₁ and R₂ or R′₂, R₅ or R′₅ and R₆ or R′₆, R₉ or R′₉ and R₁₀ together with the carbon atoms to which they are attached independently form a substituted or unsubstituted and saturated, partially saturated, or unsaturated cycle or heterocycle having 3 to 20 carbon atoms; and
(v) optionally, one or more of R₂ and R′₂, R₅ and R′₅, R₆ and R′₆, and R₉ and R′₉, together with the carbon atom to which they are attached independently form a substituted or unsubstituted and saturated, partially saturated, or unsaturated cycle or heterocycle having 3 to 20 carbon atoms; and
(vi) optionally, one or more of R₂ or R′₂ and R₃, R₄ and R₅ or R′₅, R₆ or R′₆ and R₇, or R₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(vii) optionally, one or more of R₁, R₂, R′₂, R₃, R₄, R₅, R′₅, R₆, R′₆, R₇, R₈, R₉, R′₉, and R₁₀, together with a different one of R₁, R₂, R′₂, R₃, R₄, R₅, R′₅, R₆, R′₆, R₇, R₈, R₉, R′₉, and R₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(viii) optionally, one or more of R₁, R₂, R′₂, R₃, R₄, R₅, R′₅, R₆, R′₆, R₇, R₈, R₉, R′₉, and R₁₀, may be individually bound to an atom of heterocycles U, V and W to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(ix) combinations of any of (i) through (viii) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R₂, R′₂, R₃, R₄, R₅, R′₅, R₆, R′₆, R₇, R₈, R₉, R′₉, and R₁₀; and
n is an integer from 0 to 3.

9. A method according to claim 8, wherein M is selected from the group consisting of Mn, Fe, Ni, Cu and V.

10. A method according to claim 8, wherein U and V are saturated cycloalkyl heterocycles having 3 to 20 carbon atoms.

11. A method according to claim 8, wherein U and V are saturated cycloalkyl heterocycles having 4 to 10 carbon atoms.

12. A method according to claim 8, wherein U and V are trans-cyclohexanyl fused rings.

13. A method according to claim 8, wherein W is a substituted or unsubstituted pyridino moiety.

14. A method according to claim 8, wherein U and V are trans-cyclohexanyl fused rings and W is a substituted pyridino moiety.

15. A method according to claim 2, wherein the superoxide dismutase mimetic is represented by the formula:

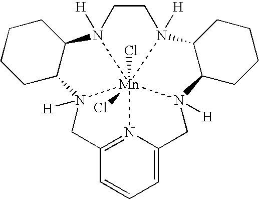

16. A method according to claim 1, wherein the ROS scavenger is a peroxynitrite scavenger.

17. A method according to claim 16, wherein the peroxynitrite scavenger is represented by a formula selected from the group of formulas consisting of: 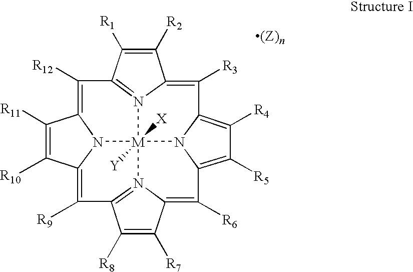
wherein R₃, R₆, R₉ and R₁₂ are independently selected from the group consisting of H, alkyl, alkenyl, CH₂, COOH, phenyl, pyridyl, and N-alkylpyridyl, such that phenyl, pyridyl and N-alkylpyridyl are: 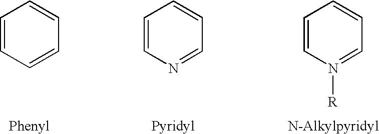
which are attached at a carbon atom; and
wherein Phenyl is optionally substituted by a substituent selected from the group consisting of a halogen, alkyl, aryl, benzyl, COOH, CONH₂, SO₃H, NO₂, NH₂, N(R)³⁺ and NHCOR′, wherein R is selected from the group consisting of hydrogen, alkyl, aryl and alkaryl, and R′ is alkyl:
wherein Pyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and
wherein N-Alkylpyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and
wherein R₁, R₂, R₄, R₅, R₇, R₈, R₁₀, or R₁₁ are independently selected from the group consisting of H, alkyl, alkenyl, carboxyalkyl, Cl, Br, F, NO₂, hydroxyalkyl, and SO₃H; and further wherein R₁ and R₂ optionally form a heterocycle having 5 to 8 carbon atoms and form a ring with the carbon atoms of the macrocycle to which they are attached;
X and Y are ligands or charge-neutralizing anions which are derived from any monodentate or polydentate coordinating ligand or ligand system or the corresponding anion thereof and are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl, amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl, phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluomanitmonate, hypophosphite, iodate, periodate, metaborate, tetraarylborate, tetra alkyl borate, tartrate, salicylate, succinate, citrate, ascorbate, saccharinate, amino acid, hydroxamic acid, thiotosylate, and anions of ion exchange resins; with the proviso that when the X and Y containing complex has a net positive charge then Z is a counter ion which is independently selected from the group consisting of X and Y, or when the X and Y containing complex has net negative charge then Z is a counter ion selected from a group consisting of alkaline and alkaline earth cations organic cations such as alkyl or alkylaryl ammonium cations; and
M is selected from the group consisting of Mn, Fe, Ni and V; and
n is an integer from 0 to 4. 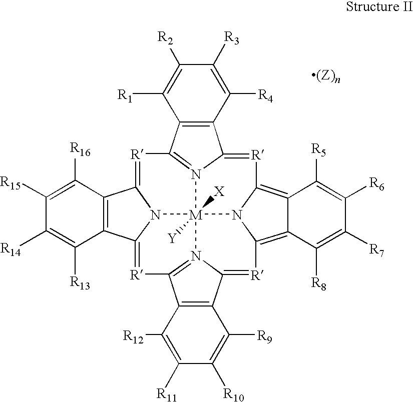
wherein R′ is CH or N;
R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of H, SO₃H, COOH, NO₂, NH₂, and N-alkylamino; and
X, Y, Z, M and n are as defined above; 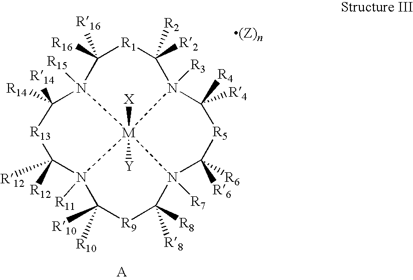
wherein R₁, R₅, R₉, and R₁₃ are independently selected from the group consisting of a direct bond and CH₂;
R₂, R′₂, R₄, R′₄, R₆, R′₆, R₈, R′₈, R₁₀, R′₁₀, R₁₂, R′₁₂, R′₄, R′₁₄, R₁₆, and R′₁₆ are independently selected from the group consisting of H and alkyl;
R₃, R₇, R₁₁, and R₁₅ are independently selected from the group consisting of Hand alkyl; and
X, Y, Z, M and n areas defined above; 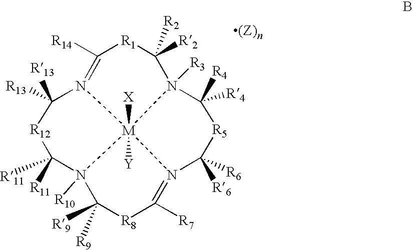
wherein R₁, R₅, R₈, and R₁₂ are independently selected from the group consisting of a direct bond and CH₂;
R₂, R′₂, R₄, R′₄, R₆, R′₆, R₇, R₉, R′₉, R₁₁, R′₁₁, R₁₃, R′₁₃, and R₁₄ are independently selected from the group consisting of H and alkyl;
R₃ and R₁₀ are independently selected from the group consisting of H and alkyl; and
X, Y, Z, M and n are as defined above; 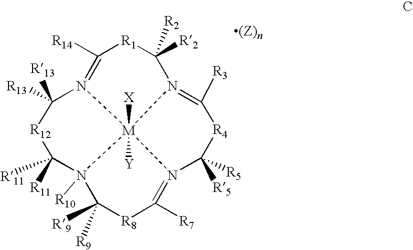
wherein R₁, R₄, R₈, and R₁₂ are independently selected from the group consisting of a direct bond and CH₂;
R₂, R′₂, R₃, R₅, R′₅, R₇, R₉, R′₉, R₁₁, R′₁₁, R₁₃, R′₁₃ and R₁₄ are independently selected from the group consisting of H and alkyl;
R₁₀ is H or alkyl; and
X, Y, Z, M and n are as defined above; 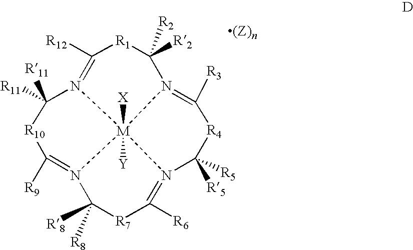
wherein R₁, R₄, R₇ and R₁₀ are independently selected from the group consisting of a direct bond and CH₂;
R₂, R′₂, R₃, R₅, R′₅, R₆, R₈, R′₈, R₉, R₁₁, R′₁₁ and R₁₂ are independently selected from the group consisting of H and alkyl; and
X, Y, Z, M and n are as defined above; 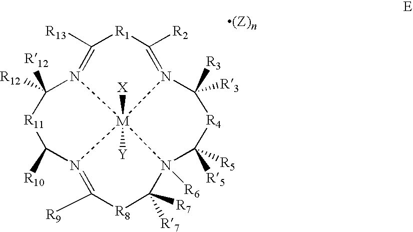
wherein R₁, R₄, R₈ and R₁₁ are independently selected from the group consisting of a direct bond and CH₂;
R₂, R₃, R′₃, R₅, R′₅, R₇, R′₇, R₉, R₁₀, R′₁₀, R₁₂, R′₁₂ and R₁₃ are independently selected from the group consisting of H and alkyl;
R is hydrogen or alkyl; and
X, Y, Z, M and n are as defined above; 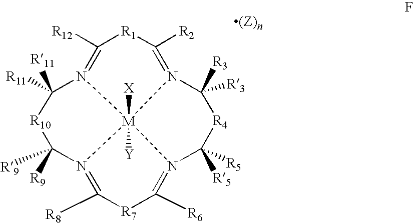
wherein R₁, R₄, R₇ and R₁₀ are independently selected from the group consisting of H and alkyl:
R₂, R₃, R′₃, R₅, R′₅, R₆, R₈, R₉, R′₉, R₁₁, R′₁₁ and R₁₂ are independently selected from the group consisting of H and alkyl; and
X, Y, Z, M and n are as defined above; 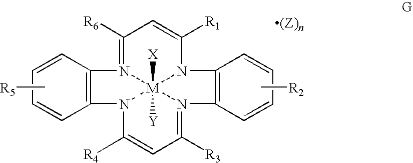
wherein R₁, R₃, R₄, and R₆ are independently selected from the group consisting of H and alkyl;
R₂ and R₅ are independently selected from the group consisting of H, alkyl, SO₃H, NO₂, NH₂, halogen, COOH and N(R)³⁺ wherein R is as defined above; and
X, Y, Z, M and n are as defined above; 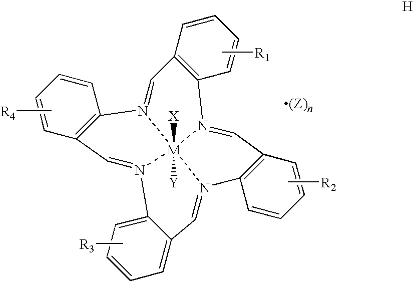
wherein R₁, R₂, R₃, and R₄ are independently selected from the group consisting of H, alkyl, SO₃H, NO₂, NH₂, halogen, COOH and N(R)³⁺ wherein R is as defined above; and
X, Y, Z, M and n are as defined above; and 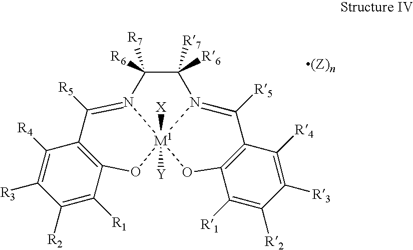
wherein R₁, R′₁, R₂, R′₂, R₃, R′₃, R₄, R′₄, R₅, R′₅, R₆, R′₆, R₇ and R′₇ are independently selected from the group consisting of H, alkyl, alkoxy, NO₂, aryl, halogen, NH₂ and SO₃H, further wherein R₆, R′₆, R₇ and R′₇ together with one other of R₆, R′₆, R₇ and R′₇ optionally form a heterocycle having 5 to 8 carbon atoms and form a ring with the carbon atoms of the macrocycle to which they are attached;
M¹ is selected from the group consisting of Fe, Ni or V; and
X, Y, Z and n are as defined above.

18. A method according to claim 1, wherein the methotrexate and the ROS scavenger are administered in amounts that act synergistically in treating the inflammatory disease.

19. A method according to claim 18, wherein the methotrexate is administered in an amount of at most 0.015 mg/kg.

20. A method according to claim 18, wherein the superoxide dismutase mimetic is administered in an amount of at most 2 mg/kg.

21. A method according to claim 1, wherein the pharmaceutically acceptable formulation is a pharmaceutically acceptable oral formulation and administering comprises administering orally.

22. A method according to claim 1, wherein the patient is a human patient.

23. A method according to claim 1, wherein the inflammatory disease is rheumatoid arthritis, psoriasis, inflammatory bowel disease or corticosteroid-dependent asthma.

24. A method according to claim 1, wherein the inflammatory disease is rheumatoid arthritis.

25. A method according to claim 1, wherein the methotrexate and the ROS scavenger are administered in one compound.

26. A method according to claim 1, wherein the methotrexate and the ROS scavenger are administered as separate compositions.

27. A pharmaceutical compound for treating an inflammatory disease, the compound comprising methotrexate and a ROS scavenger in a pharmaceutically acceptable formulation containing at least one unit dose suitable for administration to a patient in need thereof.

28. A compound according to claim 27, wherein the ROS scavenger is a superoxide dismutase mimetic.

29. A compound according to claim 28, wherein the superoxide dismutase mimetic is represented by the formula: 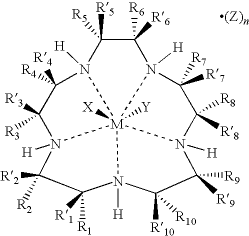
wherein
(i) R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀ are independently:
(i^{a}) hydrogen; or
(i^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(i^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(ii) optionally, one or more of R₁ or R′₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ or R′₁₀ together with the carbon atoms to which they are attached independently form a substituted or unsubstituted and saturated, partially saturated, or unsaturated cycle or heterocycle having 3 to 20 carbon atoms; and
(iii) optionally, one or more of R₁ and R′₁, R₂ and R′₂, R₃ and R′₃, R₄ and R′₄, R₅ and R′₅, R₅ and R′₆, R₇ and R′₇, R₈ and R′₈, R₉ and R′₉, and R₁₀ and R′₁₀, together with the carbon atom to which they are attached independently form a substituted or unsubstituted and saturated, partially saturated, or unsaturated cycle or heterocycle having 3 to 20 carbon atoms; and
(iv) optionally, one or more of R₁₀ or R′₁₀ and R₁ or R′₁, R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(v) optionally, one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, together with a different one of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vi) combinations of any of (i) through (v) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrite, aryl nitrite, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra-alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R′₁, R₂, R′₂, R₃, R′₃, R₄, R′₄, R₅, R′₅, R₆, R′₆, R₇, R′₇, R₈, R′₈, R₉, R′₉, R₁₀, and R′₁₀; and
n is an integer from 0 to 3.

30. A compound according to claim 28, wherein M is selected from the group consisting of Mn, Fe, Ni, Cu and V.

31. A compound according to claim 28, wherein the superoxide dismutase mimetic is represented by the formula: 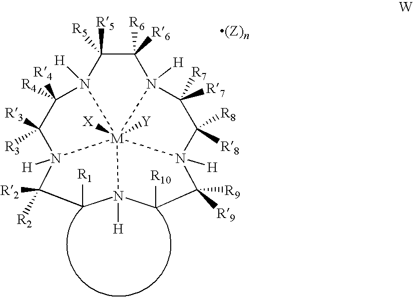
wherein
(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(ii) one or more of R₁, R₂, R′₂, R₃, R′₃, R₄, R′₄, R₅, R′₅, R₆, R′₆, R₇, R′₇, R₈, R′₈, R₉, R′₉, and R₁₀ are independently:
(ii^{a}) hydrogen; or
(ii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(ii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(iii) optionally, one or more of R₁ and R₂ or R′₂, R₃ or R′₃ and R₄ or R′₄, R₅ or R′₅ and R₆ or R′₆, R₇ or R′₇ and R₈ or R′₈, R₉ or R′₉ and R₁₀ together with the carbon atoms to which they are attached independently form a substituted or unsubstituted and saturated, partially saturated, or unsaturated cycle or heterocycle having 3 to 20 carbon atoms; and
(iv) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a substituted or unsubstituted and saturated, partially saturated, or unsaturated cycle or heterocycle having 3 to 20 carbon atoms; and
(v) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(vi) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, together with a different one of R₁, R₂, R′₂, R₃, R′₃, R₄, R′₄, R₅, R′₅, R₆, R′₆, R₇, R′₇, R₈, R′₈, R₉, R′₉, and R₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(vii) optionally, one or more of R₁, R₂, R′₂, R₃, R′₃, R₄, R′₄, R₅, R′₅, R₆, R′₆, R₇, R′₇, R₈, R′₈, R₉, R′₉, and R₁₀, may be bound to an atom of heterocycle W to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(viii) combinations of any of (i) through (vii) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl, carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R₂, R′₂, R₃, R′₃, R₄, R′₄, R₅, R′₅, R₆, R′₆, R₇, R′₇, R₈, R′₈, R₉, R′₉, and R₁₀; and
n is an integer from 0 to 3.

32. A compound according to claim 31., wherein M is selected from the group consisting of Mn, Fe, Ni, Cu and V.

33. A compound according to claim 31, wherein W is a substituted or unsubstituted pyridino moiety.

34. A compound according to claim 28, wherein the superoxide dismutase mimetic is represented by the formula: 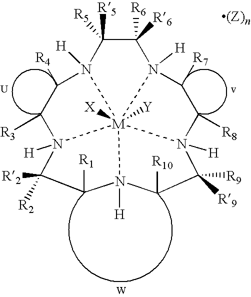
wherein
(i) a nitrogen of the macrocycle and two adjacent carbon atoms to which the nitrogen is attached independently form a substituted or unsubstituted, saturated, partially saturated or unsaturated nitrogen-containing heterocycle W having 2 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(ii) two sets of two adjacent carbon atoms of the macrocycle independently form substituted or unsubstituted, saturated, partially saturated or unsaturated, cycles or heterocycles U and V having 3 to 20 carbon atoms; and
(iii) R₁, R₂, R′₂, R₃, R₄, R₅, R′₅, R₆, R′₆, R₇, R₈, R₉, R′₉, and R₁₀ are independently:
(iii^{a}) hydrogen; or
(iii^{b}) a moiety independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl; or
(iii^{c}) a moiety independently selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), —OP(O)(OR₁₁)(OR₁₂), and substituents attached to the α-carbon of α-amino acids, wherein R₁₁ and R₁₂ are independently hydrogen or alkyl; and
(iv) optionally, one or more of R₁ and R₂ or R′₂, R₅ or R′₅ and R₆ or R′₆, R₉ or R′₉ and R₁₀ together with the carbon atoms to which they are attached independently form a substituted or unsubstituted and saturated, partially saturated, or unsaturated cycle or heterocycle having 3 to 20 carbon atoms; and
(v) optionally, one or more of R₂ and R′₂, R₅ and R′₅, R₆ and R′₆, and R₉ and R′₉, together with the carbon atom to which they are attached independently form a substituted or unsubstituted and saturated, partially saturated, or unsaturated cycle or heterocycle having 3 to 20 carbon atoms; and
(vi) optionally, one or more of R₂ or R′₂ and R₃, R₄ and R₅ or R′₅, R₆ or R′₆ and R₇, or R₈ and R₉ or R′₉ together with the carbon atoms to which they are attached independently form a substituted or unsubstituted nitrogen containing heterocycle having 3 to 20 carbon atoms, which may be an aromatic heterocycle in which case the hydrogen attached to the nitrogen which is both part of the heterocycle and the macrocycle and the R groups attached to the carbon atoms which are both part of the heterocycle and the macrocycle are absent; and
(vii) optionally, one or more of R₁, R₂, R′₂, R₃, R₄, R₅, R′₅, R₆, R′₆, R₇, R₈, R₉, R′₉, and R₁₀, together with a different one of R₁, R₂, R′₂, R₃, R₄, R₅, R′₅, R₆, R′₆, R₇, R₈, R₉, R′₉, and R₁₀, which is attached to a different carbon atom in the macrocyclic ligand may be bound to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(viii) optionally, one or more of R₁, R₂, R′₂, R₃, R₄, R₅, R′₅, R₆, R′₆, R₇, R₈, R₉, R′₉, and R₁₀, may be individually bound to an atom of heterocycles U, V and W to form a strap represented by the formula:
—(CH₂)_{I}-Q-(CH₂)_{J}—R—(CH₂)_{K}—S—(CH₂)_{L}—
wherein
I, J, K and L independently are integers from 0 to 10 and Q, R and S are independently selected from the group consisting of alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and heterocyclyl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza, and combinations thereof; and
(ix) combinations of any of (i) through (viii) above;
wherein
M is a transition metal;
X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof; or
X, Y and Z are independently selected from the group consisting of charge-neutralizing anions which are derived from any monodentate or polydentate coordinating ligand and a ligand system and the corresponding anion thereof; or
X, Y and Z are independently attached to one or more of R₁, R₂, R′₂, R₃, R₄, R₅, R′₅, R₆, R′₆, R₇, R₈, R₉, R′₉, and R₁₀; and
n is an integer from 0 to 3.

35. A compound according to claim 34, wherein M is selected from the group consisting of Mn, Fe, Ni, Cu and V.

36. A compound according to claim 34, wherein U and V are saturated cycloalkyl heterocycles having 3 to 20 carbon atoms.

37. A compound according to claim 34, wherein U and V are saturated cycloalkyl heterocycles having 4 to 10 carbon atoms.

38. A compound according to claim 34, wherein U and V are trans-cyclohexanyl fused rings.

39. A compound according to claim 34, wherein W is a substituted or unsubstituted pyridino moiety.

40. A compound according to claim 34, wherein U and V are trans-cyclohexanyl fused rings and W is a substituted pyridino moiety.

41. A compound according to claim 28, wherein the superoxide dismutase mimetic is represented by the formula:

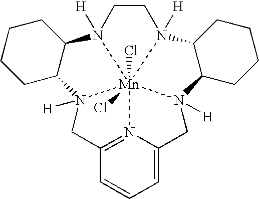

42. A compound according to claim 27, wherein the ROS scavenger is a peroxynitrite scavenger.

43. A compound according to claim 42, wherein the peroxynitrite scavenger is represented by a formula selected from the group of formulas consisting of: 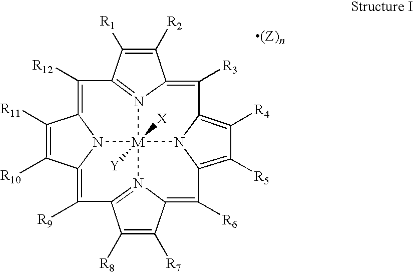
wherein R₃, R₆, R₉ and R₁₂ are independently selected from the group consisting of H, alkyl, alkenyl, CH₂, COOH, phenyl, pyridyl, and N-alkylpyridyl, such that phenyl, pyridyl and N-alkylpyridyl are: 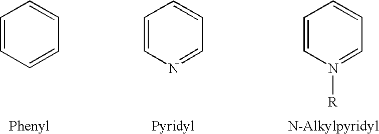
which are attached at a carbon atom; and
wherein Phenyl is optionally substituted by a substituent selected from the group consisting of a halogen, alkyl, aryl, benzyl, COOH, CONH₂, SO₃H, NO₂, NH₂, N(R)³⁺ and NHCOR′, wherein R is selected from the group consisting of hydrogen, alkyl, aryl and alkaryl, and R′ is alkyl;
wherein Pyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and
wherein N-Alkylpyridyl is optionally substituted by a substituent selected from the group consisting of a halogen, alkyl, aryl, benzyl, COOH, CONH₂, SO₃H, NO₂, NH₂, N(R)³⁺ and NHCOR′, wherein R and R′ are as defined above; and
wherein R₁, R₂, R₄, R₅, R₇, R₈, R₁₀, or R₁₁ are independently-selected from the group consisting of H, alkyl, alkenyl, carboxyalkyl, Cl, Br, F, NO₂, hydroxyalkyl, and SO₃H; and further wherein R₁ and R₂ optionally form a heterocycle having 5 to 8 carbon atoms and form a ring with the carbon atoms of the macrocycle to which they are attached;
X and Y are ligands or charge-neutralizing anions which are derived from any monodentate or polydentate coordinating ligand or ligand system or the corresponding anion thereof and are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl, amino, amine oxides, hydrazine, alkyl hydrazine, aryl hydrazine, nitric oxide, cyanide, cyanate, thiocyanate; isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroanitmonate, hypophosphite, Iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins; with the proviso that when the X and Y containing complex has a net positive charge then Z is a counter ion which is independently selected from the group consisting of X and Y, or when the X and Y containing complex has net negative charge then Z is a counter ion selected from a group consisting of alkaline and alkaline earth cations, organic cations such as alkyl or alkylaryl ammonium cations; and
M is selected from the group consisting of Mn, Fe, Ni and V; and
n is an integer from 0 to 4. 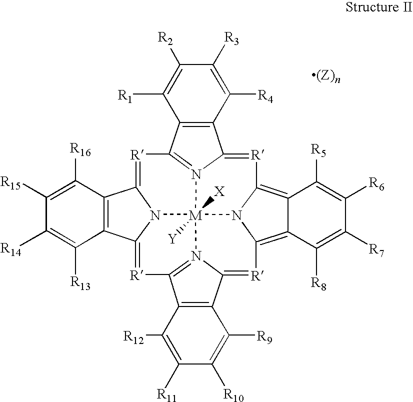
wherein R′ is CH or N,
R₁, R₂, R₃, R₄, R₅, R₆, R₇, R₈, R₉, R₁₀, R₁₁, R₁₂, R₁₃, R₁₄, R₁₅, and R₁₆ are independently selected from the group consisting of H, SO₃H, COOH, NO₂, NH₂, and N-alkylamino; and
X, Y, Z, M and n are as defined above; 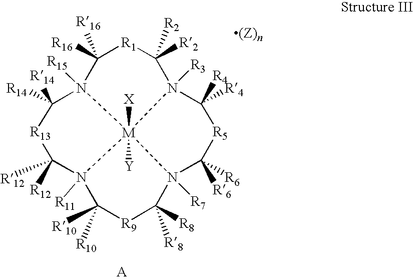
wherein R₁, R₅, R₉, and R₁₃ are independently selected from the group consisting of a direct bond and CH₂;
R₂, R′₂, R₄, R′₄, R₆, R′₆, R₈, R′₈, R₁₀, R′₁₀, R₁₂, R′₁₂, R₁₄, R′₁₄, R₁₆, and R′₁₆ are independently selected from the group consisting of H and alkyl;
R₃, R₇, R₁₁, and R₁₅ are independently selected from the group consisting of H and alkyl; and
X, Y, Z, M and n are as defined above; 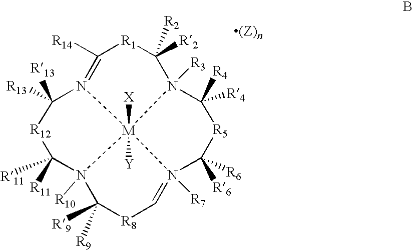
wherein R₁, R₅, R₈, and R₁₂ are independently selected from the group consisting of a direct bond and CH₂;
R₂, R′₂, R₄, R′₄, R₆, R′₆, R₇, R₉, R′₉, R₁₁, R′₁₁, R₁₃, R′₁₃, and R₁₄ are independently selected from the group consisting of H and alkyl;
R₃ and R₁₀ are independently selected from the group consisting of H and alkyl; and
X, Y, Z, M and n are as defined above; 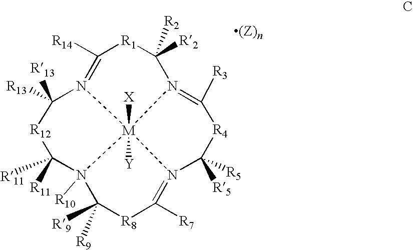
wherein R₁, R₄, R₈, and R′₂ are independently selected from the group consisting of a direct bond and CH₂;
R₂, R′₂, R₃, R₅, R′₅, R₇, R₉, R′₉, R₁₁, R′₁₁, R₁₃, R′₁₃ and R₁₄ are independently selected from the group consisting of H and alkyl;
R₁₀ is H or alkyl; and
X, Y, Z, M and n are as defined above; 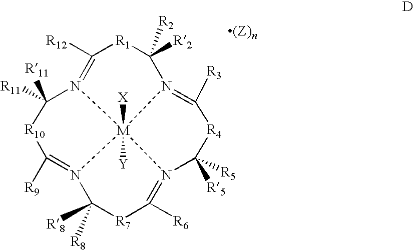
wherein R₁, R₄, R₇ and R₁₀ are independently selected from the group consisting of a direct bond and CH₂;
R₂, R′₂, R₃, R₅, R′₅, R₆, R₈, R′₈, R₉, R₁₁, R′₁₁, and R₁₂ are independently selected from the group consisting of H and alkyl; and
X, Y, Z, M and n are as defined above; 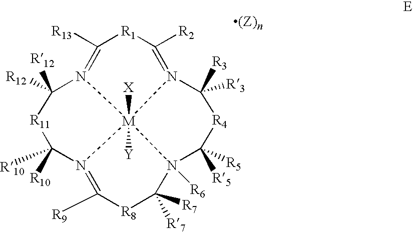
wherein R₁, R₄, R₈ and R₁₁ are independently selected from the group consisting of a direct bond and CH₂;
R₂, R₃, R′₃, R₅, R′₅, R₇, R′₇, R₉, R₁₀, R′₁₀, R₁₂, R′₁₂ and R₁₃ are independently selected from the group consisting of H and alkyl;
R₆ is hydrogen or alkyl; and
X, Y, Z, M and n are as defined above; 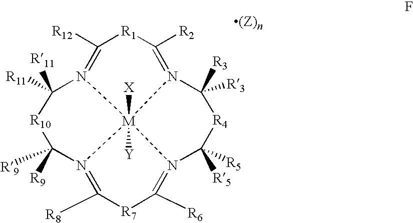
wherein R₁, R₄, R₇ and R₁₀ are independently selected from the group consisting of H and alkyl;
R₂, R₃, R′₃, R₅, R′₅, R₆, R₈, R₉, R′₉, R₁₁, R′₁₁ and R₁₂ are independently selected from the group consisting of H and alkyl; and
X, Y, Z, M and n are as defined above; 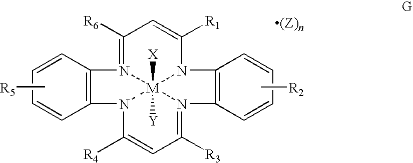
wherein R₁, R₃, R₄, and R₆ are independently selected from the group consisting of H and alkyl;
R₂ and R₅ are independently selected from the group consisting of H, alkyl, SO₃H, NO₂, NH₂, halogen, COOH and N(R)³⁺ wherein R is as defined above; and
X, Y, Z, M and n are as defined above; 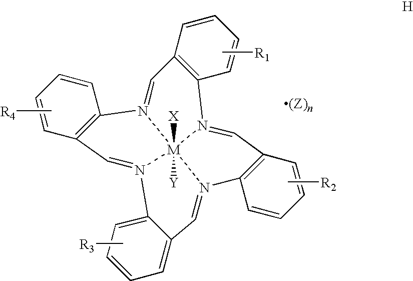
wherein R₁, R₂, R₃, and R₄ are independently selected from the group consisting of H, alkyl, SO₃H, NO₂, NH₂, halogen, COOH and N(R)³⁺ wherein R is as defined above; and
X, Y, Z, M and n are as defined above; and 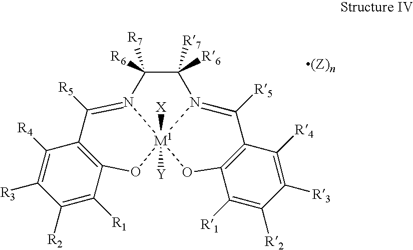
wherein R₁, R′₁, R₂, R′₂, R₃, R′₃, R₄, R′₄, R₅, R′₅, R₆, R′₆, R₇ and R′₇ are independently selected from the group consisting of H, alkyl, alkoxy, NO₂, aryl, halogen, NH₂ and SO₃H, further wherein R₆, R′₆, R₇ and R′₇ together with one other of R₆, R′₆, R₇ and R′₇ optionally form a heterocycle having 5 to 8 carbon atoms and form a ring with the carbon atoms of the macrocycle to which they are attached;
M¹ is selected from the group consisting of Fe, Ni or V; and
X, Y, Z and n are as defined above.

44. A compound according to claim 27, wherein the at least one unit dose comprises methotrexate and the ROS scavenger in amounts that act synergistically in treating the inflammatory disease.

45. A compound according to claim 27, wherein the unit dose comprises methotrexate in an amount of at most 0.015 mg/kg.

46. A compound according to claim 27, wherein the unit dose comprises the superoxide dismutase mimetic in an amount of at most 2 mg/kg.

47. A compound according to claim 27, wherein the pharmaceutically acceptable formulation is a pharmaceutically acceptable oral formulation.

48. A compound according to claim 27, wherein the patient is a human patient.

49. A compound according to claim 27, wherein the Inflammatory disease is rheumatoid arthritis, psoriasis, inflammatory bowel disease or corticosteroid-dependent asthma.

50. A compound according to claim 27, wherein the inflammatory disease is rheumatoid arthritis.

51. A kit for treating an inflammatory disease, the kit comprising methotrexate and a ROS scavenger, wherein each of the methotrexate and the ROS scavenger are present in a pharmaceutically acceptable formulation containing at least one unit dose suitable for administration to a patient in need thereof.
